(19) United States
(12) Patent Application Publication
(10) Pub. No.: US 2023/0043569 A1
(43) Pub. Date: Feb. 9, 2023

(54) PRODUCTION OF GPP AND CBGA IN A METHYLOTROPHIC YEAST STRAIN

(71) Applicant: Exponential Genomics Canada Inc., Vancouver (CA)

(72) Inventors: David F. Preiner, Providence, RI (US); Casey W. Solomon, St. Paul, MN (US); Sisi Ni, Somerville, MA (US); John W. Harrold, JR., Somerville, MA (US)

(73) Assignee: Exponential Genomics Canada Inc., Vancouver (CA)

(21) Appl. No.: 17/683,123

(22) Filed: Feb. 28, 2022

Related U.S. Application Data
(63) Continuation of application No. PCT/CA20/51176, filed on Aug. 28, 2020.
(60) Provisional application No. 62/899,948, filed on Sep. 13, 2019.
(60) Provisional application No. 62/894,146, filed on Aug. 30, 2019.

(51) Int. Cl. A61K 31/352; C12N 15/81; C12N 1/16; C12N 9/86; C12N 9/90

(52) U.S. Cl. CPC A61K 31/352; C12N 15/81; C12N 1/165; C12N 9/86; C12N 9/90; C12R 2001/84

(57) ABSTRACT

This invention is an improved method of robust and scalable production of precursors of active cannabinoids, including geranyl pyrophosphate (GPP) and/or cannabigerolic acid (CBGA), in a methylotrophic yeast host cell. The improved methods incorporate a polypeptide encoding an Erg20 variant (F98W/N128W) into a methylotrophic yeast host cell, for example Pichia pastoris (Komagataella phaffii), that biases the natural production of FPP and GPP towards GPP, a precursor to the intermediate CBGA, crucial to the synthesis of active cannabinoids.

### CROSS REFERENCE TO RELATED APPLICATIONS

This application is a continuation of International Application No. PCT/CA2020/051176, filed with the Patent Cooperation Treaty, Canadian Receiving Office on Aug. 28, 2020, which claims priority to United States Provisional Patent Application Numbers U.S. 62/894,146, filed Aug. 30, 2019, and U.S. 62/899,948, filed Sep. 13, 2019. The entirety of each of these applications is hereby incorporated by reference for all purposes.

### FIELD OF THE INVENTION

The present disclosure relates generally to production of GPP and/or cannabigerolic acid (CBGA) in a methylotrophic yeast strain containing a modified Erg20 gene.

### INCORPORATION BY REFERENCE

The contents of the text file named “22022-002WO1US1_SequenceListing”, which was created on Feb. 24, 2022, and is 26.6 KB in size, are hereby incorporated by reference in their entirety.

### BACKGROUND OF THE INVENTION

Cannabis is a genus of flowering plants that have been consumed by humans since at least 440 BCE. Cannabinoids represent a class of small molecules that interact with the human endocannabinoid system. Plant derived cannabinoids are classified as phytocannabinoids that are found in the cannabis plant as well as a variety of other plant species. Evidence suggests endogenous cannabinoids (endocannabinoids) play a critical role in regulating homeostasis in disease conditions by way of interactions with cannabinoid receptor type 1 (CB1) and cannabinoid receptor type 2 (CB2). Human cannabinoid receptors are G protein-coupled receptors (GPCR) (Mackie, 2006).

Cannabis has been investigated as a medicinal product for multiple sclerosis spasms, sleep disorders, Tourette syndrome, glaucoma, anxiety, psychosis, depression, appetite stimulation in HIV/AIDS patients, chronic pain, and nausea and vomiting due to chemotherapy treatments (doi: 10.1001/jama.2015.6358)(The Health Effects of Cannabis and Cannabinoids: The Current State of Evidence and Recommendations for Research).

Despite having a high pharmacological potential for a broad range of therapeutic applications, obtaining highly purified cannabinoids remains a significant challenge that is oftentimes extremely resource intensive and therefore cost prohibitive. Plant extraction can be an especially difficult as cannabinoids are difficult to separate from other plant biomass and result in a contaminated product that is not purified to an extent necessary for most pharmaceutical applications or to meet regulatory standards of purity.

An inability to cost effectively purify individual cannabinoids from plant extracts not only makes pharmaceutical targets difficult for researchers to study and a challenge for regulators to approve, but in turn leads consumers to self-medicate with plant based Cannabis that contains unknown chemical compositions. Cannabis sativa strains can vary widely in the level of composition of constituent cannabinoids. Unknown specificities of chemical composition make it nearly impossible to predict the exact pharmacological effects consuming plant based Cannabis may provoke and gives little to no specificity to characterize or to target individual biochemical processes. These similar molecules can make isolation difficult. In addition to difficulties in the process of extracting cannabinoids from plants, yields of cannabinoid extract remain extremely low in compared to the overall weight of plant biomass with some sources stating cannabinoid yield extracts as low as 2.5% of total weight (In Med, 2019). Low extraction yields are partially explained by the hydrophobicity of most cannabinoids and add to the cost prohibitive nature of producing purified cannabinoid extracts from plants. Difficulties of extracting plant based cannabinoids are further exacerbated when attempting to purify individual cannabinoids.

There remains a need to produce the precursors of cannabinoids.

### SUMMARY OF THE INVENTION

In one aspect there is provided a method of producing geranyl pyrophosphate (GPP) in a methylotrophic yeast host cell, comprising:
introducing a first polynucleotide encoding an Erg20 (F98W/N128W) polypeptide,
culturing the methylotrophic yeast host cell under conditions sufficient for GPP production.

In one example, said methylotrophic yeast host cell is from Pichia Pastoris (Komagataella phaffi).

In one example, the first polynucleotide encoding an Erg20 (F98W/N128W) polypeptide comprises or consists of:

a) a nucleotide sequence as set forth in SEQ ID NO:1 or SEQ ID NO:2;

b) a nucleic acid having at least 70% identity to the nucleic acid of a),

c) a nucleic acid that hybridizes with the complementary strand of the nucleic acid of a),

d) a nucleic acid that differs from a) by one or more nucleotides that are substituted, deleted, and/or inserted; or

e) a derivative or variant of a), b), c), or d).

In one example, in step (c) said polynucleotide hybridizes with the complementary strand of the nucleic acid of a) under conditions of high stringency.

In one example there is provided an expression vector comprising an isolated polynucleotide, comprising:

a) a nucleotide sequence set forth in SEQ ID NO:1 or SEQ ID NO:2,

b) a nucleotide sequence having at least 70% identity to the nucleotide sequence of a),

c) a nucleotide sequence that hybridizes with the complementary strand of the nucleic acid of a),

d) a nucleotide sequence that differs from a) by one or more nucleotides that are substituted, deleted, and/or inserted; or

e) a derivative of a), b), c), or d).

In one example, in step (c) said polynucleotide hybridizes with the complementary strand of the nucleic acid of a) under conditions of high stringency.

In one aspect there is provided a methylotrophic yeast host cell comprising an expression vector of claim 5 or 6.

In one aspect there is provided a methylotrophic yeast host cell comprising:

a) a nucleotide sequence set forth in SEQ ID NO:1 or SEQ ID NO:2,

b) a nucleotide sequence having at least 70% identity to the nucleotide sequence of a),

c) a nucleotide sequence that hybridizes with the complementary strand of the nucleic acid of a),

d) a nucleotide sequence that differs from a) by one or more nucleotides that are substituted, deleted, and/or inserted; or

e) a derivative of a), b), c), or d).

In one example, said methylotrophic yeast host cell is from Pichia Pastoris (Komagataella phaffi).

In one aspect there is provided an isolated polynucleotide comprising or consisting of:

a) a nucleotide sequence set forth in SEQ ID NO:1 or SEQ ID NO:2,

b) a nucleotide sequence having at least 70% identity to the nucleotide sequence of a),

c) a nucleotide sequence that hybridizes with the complementary strand of the nucleic acid of a),

d) a nucleotide sequence that differs from a) by one or more nucleotides that are substituted, deleted, and/or inserted; or

e) a derivative of a), b), c), or d).

In one example, isolated polynucleotide of claim 10, wherein said polynucleotide encodes an Erg20 (F98W/N128W) polypeptide.

In one aspect there is provided a method of producing cannabigerolic acid (CBGA), in a methylotrophic yeast host cell, comprising:
introducing a first polynucleotide encoding an olevitiolic synthase polypeptide,
introducing a second polynucleotide encoding an olevitiolic acid cyclase polypeptide, and
introducing a third polynucleotide encoding an aromatic prenyl transferase,
culturing the methylotrophic yeast host cell under conditions sufficient for CBGA production.

In one aspect there is provided a method of producing cannabigerolic acid (CBGA), in a methylotrophic yeast host cell, comprising:
introducing a fourth polynucleotide encoding an olevitiolic synthase polypeptide and encoding olevitiolic acid cyclase polypeptide, and
introducing a third polynucleotide encoding an aromatic prenyl transferase,
culturing the methylotrophic yeast host cell under conditions sufficient for CBGA production.

In one aspect there is provided a method of producing cannabigerolic acid (CBGA), in a methylotrophic yeast host cell, comprising:
introducing a first polynucleotide encoding an olevitiolic synthase polypeptide, and
introducing a fifth polynucleotide encoding olevitiolic acid cyclase polypeptide and encoding acormatic prenyl transferase,
culturing the methylotrophic yeast host cell under conditions sufficient for CBGA production.

In one example, said methylotrophic yeast host cell is from Pichia Pastoris (Komagataella phaffii).

In one example, said conditions sufficient for CBGA production comprise methanol induction.

In one example, said first polynucleotide encoding olevitiolic synthase polypeptide comprises or consists of:

a) a nucleotide sequence as set forth in (SEQ ID NO:6);

b) a nucleic acid having at least 70% identity to the nucleic acid of a),

c) a nucleic acid that hybridizes with the complementary strand of the nucleic acid of a),

d) a nucleic acid that differs from a) by one or more nucleotides that are substituted, deleted, and/or inserted; or

e) a derivative of a), b), c), or d).

In one example, said second polynucleotide encoding olevitiolic acid cyclase polypeptide comprises or consists of:

a) a nucleotide sequence as set forth in (SEQ ID NO:9);

b) a nucleic acid having at least 70% identity to the nucleic acid of a),

c) a nucleic acid that hybridizes with the complementary strand of the nucleic acid of a),

d) a nucleic acid that differs from a) by one or more nucleotides that are substituted, deleted, and/or inserted; or

e) a derivative of a), b), c), or d).

in one example, said third polynucleotide encoding said acormatic prenyl transferase comprises or consists of:

a) a nucleotide sequence as set forth in (SEQ ID NO:8);

b) a nucleic acid having at least 70% identity to the nucleic acid of a),

c) a nucleic acid that hybridizes with the complementary strand of the nucleic acid of a),

d) a nucleic acid that differs from a) by one or more nucleotides that are substituted, deleted, and/or inserted; or

e) a derivative of a), b), c), or d).

In one example, said fourth polynucleotide encoding olevitiolic synthase polypeptide and encoding olevitiolic acid cyclase polypeptide comprises or consists of:

a) a nucleotide sequence as set forth in (SEQ ID NO:3);

b) a nucleic acid having at least 70% identity to the nucleic acid of a),

c) a nucleic acid that hybridizes with the complementary strand of the nucleic acid of a),

d) a nucleic acid that differs from a) by one or more nucleotides that are substituted, deleted, and/or inserted; or

e) a derivative of a), b), c), or d).

In one example said fifth polynucleotide encoding olevitiolic acid cyclase polypeptide and encoding aromatic prenyl transferase comprises or consists of:

a) a nucleotide sequence as set forth in (SEQ ID NO:10);

b) a nucleic acid having at least 70% identity to the nucleic acid of a),

c) a nucleic acid that hybridizes with the complementary strand of the nucleic acid of a),

d) a nucleic acid that differs from a) by one or more nucleotides that are substituted, deleted, and/or inserted; or

e) a derivative of a), b), c), or d).

In one example, in step (c) said polynucleotide hybridizes with the complementary strand of the nucleic acid of a) under conditions of high stringency.

In one aspect there is provided an expression vector comprising an isolated polynucleotide, comprising:

a) a nucleotide sequence set forth in SEQ ID NO:3, 6, 8, 9, or 10,

b) a nucleotide sequence having at least 70% identity to the nucleotide sequence of a),

c) a nucleotide sequence that hybridizes with the complementary strand of the nucleic acid of a),

d) a nucleotide sequence that differs from a) by one or more nucleotides that are substituted, deleted, and/or inserted; or

e) a derivative of a), b), c), or d).

In one example, in step (c) said polynucleotide hybridizes with the complementary strand of the nucleic acid of a) under conditions of high stringency.

In one aspect there is provided a methylotrophic yeast host cell comprising an expression vector of claim 23 or 24.

In one aspect there is provided methylotrophic yeast host cell comprising:

a) a nucleotide sequence set forth in SEQ ID NO:3, 6, 8, 9, or 10,

b) a nucleotide sequence having at least 70% identity to the nucleotide sequence of a),

c) a nucleotide sequence that hybridizes with the complementary strand of the nucleic acid of a),

d) a nucleotide sequence that differs from a) by one or more nucleotides that are substituted, deleted, and/or inserted; or

e) a derivative of a), b), c), or d).

In one example, said methylotrophic yeast host cell is from Pichia Pastoris (Komagataella phaffi).

### BRIEF DESCRIPTION OF THE DRAWINGS

Embodiments of the present disclosure will now be described, by way of example only, with reference to the attached Figures.

FIG. 1 depicts an Erg20 duplication vector map;

FIG. 2 depicts an Erg20 replacement vector map;

FIG. 3 depicts GPP production.

FIG. 4A-4F depicts LC-MS/MS analysis. FIG. 4A depicts Farnesyl pyrophosphate injected into water. FIG. 4B is a graph showing Peak Area versus FPP Loaded. FIG. 4C depicts Geranyl pyrophosphates injected into water. FIG. 4D is a graph showing Peak Area versus GPP Loaded. FIG. 4E shows a summary of the data. FIG. 4F shows the ratio of GPP/FPP.

FIG. 5A-5F depicts LC-MS/MS analysis. FIG. 5A depicts Farnesyl pyrophosphate injected into MeOH grown yeast. FIG. 5B is a graph showing Peak Area versus MeOH FPP Loaded. FIG. 5C depicts Geranyl pyrophosphates injected into MeOH grown in yeast. FIG. 5D is a graph showing Peak Area versus MeOH GPP Loaded. FIG. 5E shows a summary of data. FIG. 5F shows the ratio of GPP/FPP.

FIG. 6A-6E depicts LC-MS/MS analysis. FIG. 6A depicts Farnesyl pyrophosphate injected into glycerol grown yeast. FIG. 6B is a graph showing Peak Area versus glycerol FPP Loaded. FIG. 6C depicts Geranyl pyrophosphates injected into glycerol grown in yeast. FIG. 6D is a graph showing Peak Area versus glycerol GPP Loaded. FIG. 6E shows a summary of data.

FIG. 7 depicts Analytical results for CBGA production. LC/MS of GBGA generated from a P. pastoris strain that had been modified to contain the biosynthetic cannabinoid pathway. The sample was separated on a C8 reverse phase column using water and acetonitrile as aqueous and organic phases in a gradient. The detection method was a total ion current, and the CBGA peak appears at the 0.80 min mark.

FIG. 8A-8D depicts Quantification of MeOH induced P. pastoris CBGA levels. Calibration curve and analysis of LC/MS data showing the quantification of the CBGA produced in P. pastoris. FIG. 8A depicts CBGA injected into MeOH grown yeast. FIG. 8B depicts a graph of Peak Area versus MeOH CBGA. FIG. 8C shows a summary of data. FIG. 8D shows a summary of the data.

FIG. 9A-9C depicts LC-MS/MS Analysis of CBGA production. FIG. 9A depicts Cannabigerolic Acid depicted into glycerol grown in yeast. FIG. 9B depicts a graph of peak area versus glycerol CBGA loaded. FIG. 9C depicts a summary of data.

FIG. 10 depicts the pGUH CsTKS map.

FIG. 11 depicts the pJAG dual CsPT4 CsOAC map.

FIG. 12 depicts the pJUN dual CsPT4 CsOAC map.

FIG. 13 depicts the pGAH CsTKS map.

### DETAILED DESCRIPTION OF THE INVENTION

Unless otherwise defined, all terms of art, notations and other scientific terminology used herein are intended to have the meanings commonly understood by those of ordinary skill in the art to which the present application pertains. In some cases, terms with commonly understood meanings are defined herein for clarity and/or for ready reference, and the inclusion of such definitions herein should not necessarily be construed to represent a substantial difference over what is generally understood in the art.

Cannabinoids are a group of chemicals known to activate cannabinoid receptors in cells throughout the human body, including the skin. Phytocannabinoids are the cannabinoids derived from cannabis plants. Endocannabinoids are endogenous cannabinoids found in the human body.

Cannabinoids exert their physiological effects by interacting with cannabinoid receptors present on the surface of cells. To date, two types of cannabinoid receptor have been identified, the CB1 receptor and the CB2 receptor, and are distributed in different tissues and also have different signaling mechanisms. They also differ in their sensitivity to agonists and antagonists.

It is thought that cannabinoids may be used in therapeutics such as chronic pain, multiple sclerosis, cancer-associated nausea and vomiting, weight loss, appetite loss, spasticity, and other conditions.

The term cannabinoid includes acid cannabinoids and neutral cannabinoids. The term “acidic cannabinoid” refers to a cannabinoid having a carboxylic acid moiety. The carboxylic acid moiety may be present in protonated form (i.e., as —COOH) or in deprotonated form (i.e., as carboylate —COO—). Examples of acidic cannabinoids include, but are not limited to, cannabigerolic acid (CBGA), cannabidiolic acid, and A9-tetrahydrocannabinolic acid. The term “neutral cannabinoid” refers to a cannabinoid that does not contain a carboxylic acid moiety (i.e., does contain a moiety —COOH or —COO). Examples of neutral cannabinoids include, but are not limited to, cannabigerol, cannabidiol, and D9-tetrahydrocannabinol.

### Production of Geranyl Geranyl Diphosphate

In one aspect, the present disclosure provides increased production of geranyl geranyl diphosphate (also referred to as pyrophosphate GPP; C₁₀) in a methylotrophic yeast containing a modified P. pastoris Erg20 gene. In one example, the methylotrophic yeast is P. pastoris.

GPP is the diphosphate of the polyprenol compound geraniol, and is a precursor of monoterpenes, and ultimately in downstream processes in the production of cannabinoids, for example, cannabigerolic acid (CBGA).

The polypeptide Erg20 exhibits both GPP synthase (GPPS) and farnesyl pyrophosphate synthase (FPPS) activities. Erg20 condenses isopentenyl diphosphate (IPP) and dimethylallyl diphosphate (DMAP) to GPP and feranyl pyrophosphate (FPP). In the methylotrophic yeast P. pastoris, a greater amount FPP is produced than GPP in this reaction. However, as noted above, GPP is required for the production of cannabinoids, such as CBGA.

As described herein, a mutant of the Erg20 P. pastoris gene containing two point mutations (F98W and N128W) was used to increase the amount of GPP produced in P. pastoris. In one example, introduction of the mutant of the Erg20 P. pastoris gene described in to P. pastoris increases the ratio of GPP:FPP, relative to the non-mutated Erg20 gene.

Thus, in one aspect, there is described herein a metabolic engineering strategy in which a mutant of the Erg20 P. pastoris gene containing two point mutations (F98W and N128W) may be used to increase the production of GPP in P. pastoris.

While not wishing to be bound by theory, it is believed that this mutant Erg20 (F98W/N128W) from P. pastoris encodes a polypeptide that, when produced in P. pastoris, biases the natural production of FPP and GPP in P. pastoris towards GPP.

In one example, GPP production was achieved by introducing the modified Erg20 gene from P. pastoris containing two point mutations (F98W and N128W) (SEQ ID NO:1 or SEQ ID NO:2) in to P. pastoris.

In one example, GPP production was achieved by replacing the endogenous Erg20 allele of P. pastoris with the modified Erg20 gene from P. pastoris containing two point mutations (F98W and N128W) (SEQ ID NO:1).

In one example, the modified Erg20 from P. pastoris used in replacing the endogenous allele of P. pastoris comprises or consists of the following polynucleotide sequence:

(SEQ ID NO: 1)
AGTCTCAAGACTGGAGACTCGCTCTTTATAGAAATAGATATAGCCTTTTA
CTATTAATTCCATTTTAGTCATGCTGTGTTATATTTTGTAGACATGATAT
AATCACTGTATATTTTCAACTTTGATGTGCCCTTAAACGTTAACACGCTC
AAGCTCCTCTCTACCTGCCTTGGTGTACAGGTATTCATTGTAGTCTCTAG
CTTGAGCCCACACGTTCCAAACGATGATACCTGGAATAATCACATACAAA
GCCTGAGCTTTGGTTCTTCTCCAAACGTTGAAAATGGCTCTTTCCGATGA
TCCTTTCAAAGGAGATCCGACGTAAGGGGAGATGGTGTAGGTAGTGACGT
TCTTTTGCTTGGGGGATCCAATGAATCCCCACCAGCCCATGTAAGCACCT
GGATGTCCTGCCATTCTTAATGTAGTTTTAGGGTAAGTAGATGGTAGTAG
AATTTCCCGTATTATATTCGAGGCCATCTGATTGGTGATAATTCCGTTCT
GAAATATTCGCAGGGAAACTTAGGTTGGTAGCAGTGATACCTGTAACTAC
CTTGCAGAGATAATCACCAGCTAATAAAGTATGGCCTTGGCCCTTTCGCA
TAGAGAATGGTCAAGACGTACAGAACTATGAGGGCCCAACACTGTTTAAA
CTTTAGTTTGGTGCCAACTTATGATTTAAAAAGCCAGACAATTTTAATTC
CCCAAACAGACCTCTAATTTTGCCCGAATAATGCCAATTAACATTCTAGA
AACTTCGATTAACTATATATTTCATAAACCATGACCTTTTTCCGCCAATA
TCCTCATATTTCAGCCTAAGGAAGACTCTCCTCCTTTTAGCCTACAACGA
ACTTGCCCCATAAATACACCAAGCGCGACCCTCGTTCATCGTTTAACTCG
TGTTTTCTTCTCACCTTTCGAACTAGTAGAAATCAAATGTCCAAAGAAGT
AGCAGCTAAGAACAAAGTGAAGTTCCTTGCGTTCTTTCCAGACCTCGTGT
TTGAGCTGGAAGTCATGCTGGAAAATTACGGCATGCCTGCTGATGCAATC
AACTGGTTCAAGAGATCCTTGAACTACAACACTCCTGGTGGAAAACTCAA
CAGAGGTATCTCTGTTGTCGACACCTATGTCATTCTCAAGGGGTACAAGT
CATGGGACGAGTTAAGTGCCGAAGAGTTCAAGAAAGCTGCTATTTTAGGA
TGGTGTGTCGAATTGCTACAAGCCTACTGGTTGGTTGCTGATGATATGAT
GGACCAATCTATTACCAGAAGAGGTCAACCATGTTGGTACAAGGTTGAGA
ACGTCGGTAACATTGCTATTTGGGACTCTTTCATGTTGGAGGGTGCCATC
TACAAGATTTTGAGAAAGTACTTCAAGAAGGAGTCTTACTACGCCGACTT
GTTGGACTTGTTGCACGAGGTTACTTTCCAAACTGAATTGGGTCAATTGT
TGGACTTGATCACTGCTCCAGAAGACCACGTTGATCTTTCCAAGTTTACC
CCTGCTAAACACTCTTTCATTGTCATCTTCAAGACTGCCTACTATTCATT
CTACCTTCCTGTTGTTATGGCCATGTACTTGTCTGGTATTACCCATGAAA
AGGATTTGCAGCAGGCTGAGCACATCTTGATTCCATTGGGTGAGTACTTC
CAAATTCAAGATGACTACTTGGACTGTTTCGGTAAGCCAGAAGATATTGG
TAAGATTGGAACTGACATCCAGGACAATAAGTGTTCTTGGGTGATTAACC
AAGCTCTGAGATTGGCCTCTCCAGAGCAAAGACAAATTCTTGATGAGAAC
TACGGAAGAAAAGATGCCGACAAGGAGGCAAAGTGCAAGGAAGTTTTTGA
CCAACTTGACATTGCTGGTAAGTACAAGGCTTACGAGGAAAACATCGGTA
AGGAGTTGCAAAAGAGAATTGCTGACACTGAGGAGGATCGTGGATTCAAG
AAGGAAGTTTTCCAGGTCTTTTTCGACAAGATCTACAAGAGAACCAAATA
G

In one example, GPP production was achieved by introducing in to P. pastoris the modified Erg20 gene from P. pastoris containing two point mutations (F98W and N128W) (SEQ ID NO:2).

In one example, a mutant of the Erg20 P. pastoris gene containing two point mutations (F98W and N128W) comprises or consists of the following polynucleotide sequence:

(SEQ ID NO: 2)
AAGATGTTTTTGTTTAGTTGACTATAAGTTAATTTTTTATATTAATTTGC
ACTCTGTCCTTCACTAAACTTTAGTTTGGTGCCAACTTATGATTTAAAAA
GCCAGACAATTTTAATTCCCCAAACAGACCTCTAATTTTGCCCGAATAAT
GCCAATTAACATTCTAGAAACTTCGATTAACTATATATTTCATAAACCAT
GACCTTTTTCCGCCAATATCCTCATATTTCAGCCTAAGGAAGACTCTCCT
CCTTTTAGCCTACAACGAACTTGCCCCATAAATACACCAAGCGCGACCCT
CGTTCATCGTTTAACTCGTGTTAGAGACCTATTCCAGACTTCAGATCTCC
AAAAACAGGACTATATCATAATTTGGCACGTTTGAATCTTCCCCATCCTG
AAGCGGTATTTGAGATCAACTACTTCAGAGAAAACCCTCATGCTTTTTAC
ACATTGGCTGACGAACTTTACCCTGGGAGGGTCTCATTCTTCTCACCTTT
CGAACTAGTAGAAATCAAATGTCCAAAGAAGTAGCAGCTAAGAACAAAGT
GAAGTTCCTTGCGTTCTTTCCAGACCTCGTGTTTGAGCTGGAAGTCATGC
TGGAAAATTACGGCATGCCTGCTGATGCAATCAACTGGTTCAAGAGATCC
TTGAACTACAACACTCCTGGTGGAAAACTCAACAGAGGTATCTCTGTTGT
CGACACCTATGTCATTCTCAAGGGGTACAAGTCATGGGACGAGTTAAGTG
CCGAAGAGTTCAAGAAAGCTGCTATTTTAGGATGGTGTGTCGAATTGCTA
CAAGCCTACTGGTTGGTTGCTGATGATATGATGGACCAATCTATTACCAG
AAGAGGTCAACCATGTTGGTACAAGGTTGAGAACGTCGGTAACATTGCTA
TTTGGGACTCTTTCATGTTGGAGGGTGCCATCTACAAGATTTTGAGAAAG
TACTTCAAGAAGGAGTCTTACTACGCCGACTTGTTGGACTTGTTGCACGA
GGTTACTTTCCAAACTGAATTGGGTCAATTGTTGGACTTGATCACTGCTC
CAGAAGACCACGTTGATCTTTCCAAGTTTACCCCTGCTAAACACTCTTTC
ATTGTCATCTTCAAGACTGCCTACTATTCATTCTACCTTCCTGTTGTTAT
GGCCATGTACTTGTCTGGTATTACCCATGAAAAGGATTTGCAGCAGGCTG
AGCACATCTTGATTCCATTGGGTGAGTACTTCCAAATTCAAGATGACTAC
TTGGACTGTTTCGGTAAGCCAGAAGATATTGGTAAGATTGGAACTGACAT
CCAGGACAATAAGTGTTCTTGGGTGATTAACCAAGCTCTGAGATTGGCCT
CTCCAGAGCAAAGACAAATTCTTGATGAGAACTACGGAAGAAAAGATGCC
GACAAGGAGGCAAAGTGCAAGGAAGTTTTTGACCAACTTGACATTGCTGG
TAAGTACAAGGCTTACGAGGAAAACATCGGTAAGGAGTTGCAAAAGAGAA
TTGCTGACACTGAGGAGGATCGTGGATTCAAGAAGGAAGTTTTCCAGGTC
TTTTTCGACAAGATCTACAAGAGAACCAAATAG.

In one example there is provided a method of producing geranyl pyrophosphate (GPP) in a methylotrophic yeast host cell, comprising:
introducing a first polynucleotide encoding an Erg20 (F98W/N128W) polypeptide,
culturing the methylotrophic yeast host cell under conditions sufficient for GPP production.

In one example, the host cell is from the Komagataella genus.

In one example, the methylotrophic yeast host cell is Pichia Pastoris (Komagataella phaffi).

In one example, the host cell is a cell from P. pastoris.

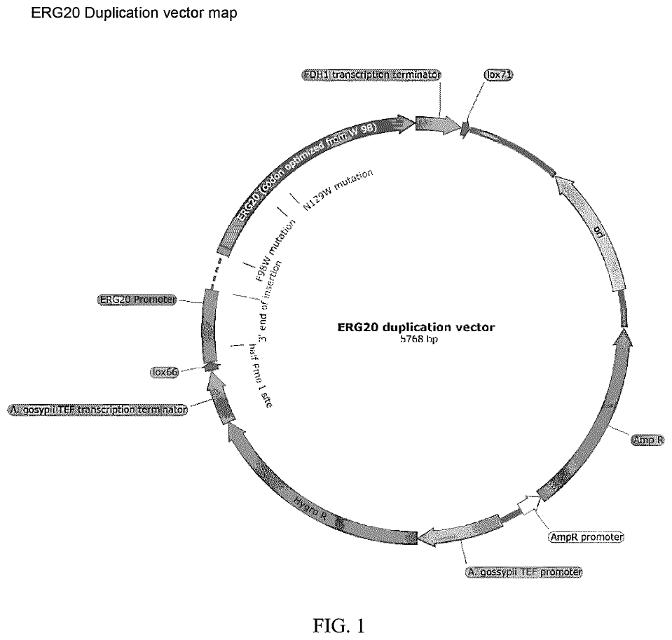

In one example, an ERG20 duplication vector map is presented in FIG. 1.

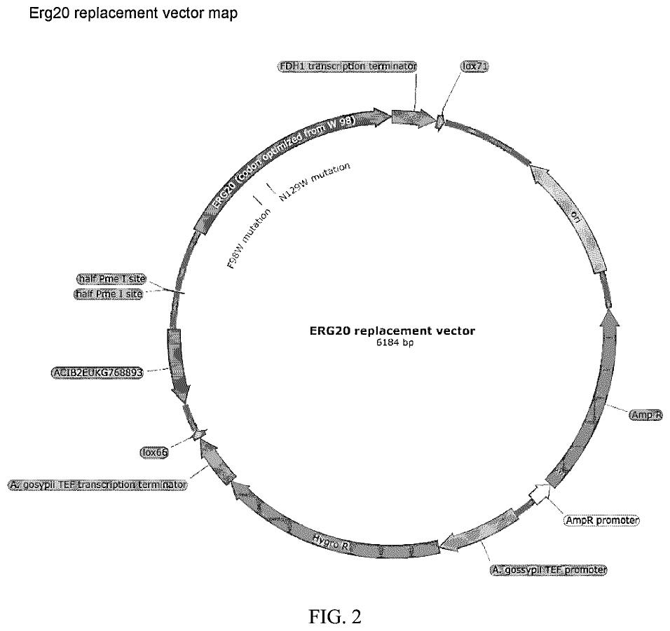

In one example, an Erg20 replacement vector map is presented in FIG. 2.

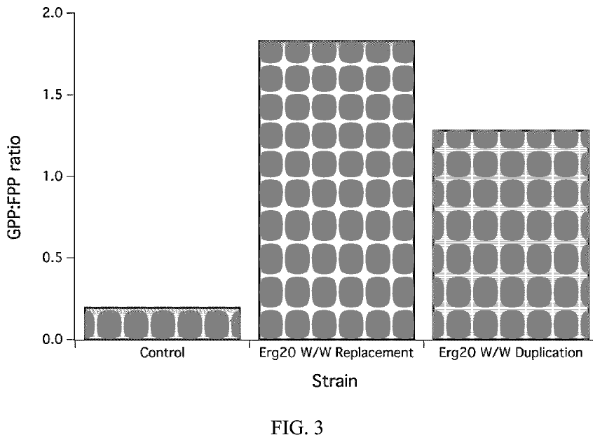

In one example, GPP production in P. pastoris is presented in FIG. 3.

In one example, the first polynucleotide encoding an Erg20 (F98W/N128W) polypeptide comprises or consists of:

a) a nucleotide sequence as set forth in (SEQ ID NO:2);

b) a nucleic acid having at least 70% identity to the nucleic acid of a),

c) a nucleic acid that hybridizes with the complementary strand of the nucleic acid of a),

d) a nucleic acid that differs from a) by one or more nucleotides that are substituted, deleted, and/or inserted; or

e) a derivative or variant of a), b), c), or d).

In one example, in step (c) said polynucleotide hybridizes with the complementary strand of the nucleic acid of a) under conditions of high stringency.

In one example, there is provided an expression vector comprising an isolated polynucleotide, comprising:

a) a nucleotide sequence set forth in SEQ ID NO: 2,

b) a nucleotide sequence having at least 70% identity to the nucleotide sequence of a),

c) a nucleotide sequence that hybridizes with the complementary strand of the nucleic acid of a),

d) a nucleotide sequence that differs from a) by one or more nucleotides that are substituted, deleted, and/or inserted; or

e) a derivative of a), b), c), or d).

In one example, the in step (c) said polynucleotide hybridizes with the complementary strand of the nucleic acid of a) under conditions of high stringency.

In one example, there is provided a methylotrophic yeast host cell comprising an expression vector of as described here.

In one example, there is provided a methylotrophic yeast host cell comprising:

a) a nucleotide sequence set forth in SEQ ID NO:2,

b) a nucleotide sequence having at least 70% identity to the nucleotide sequence of a),

c) a nucleotide sequence that hybridizes with the complementary strand of the nucleic acid of a),

d) a nucleotide sequence that differs from a) by one or more nucleotides that are substituted, deleted, and/or inserted; or

e) a derivative of a), b), c), or d).

In one example, said methylotrophic yeast host cell is from Pichia Pastoris (Komagataella phaffi).

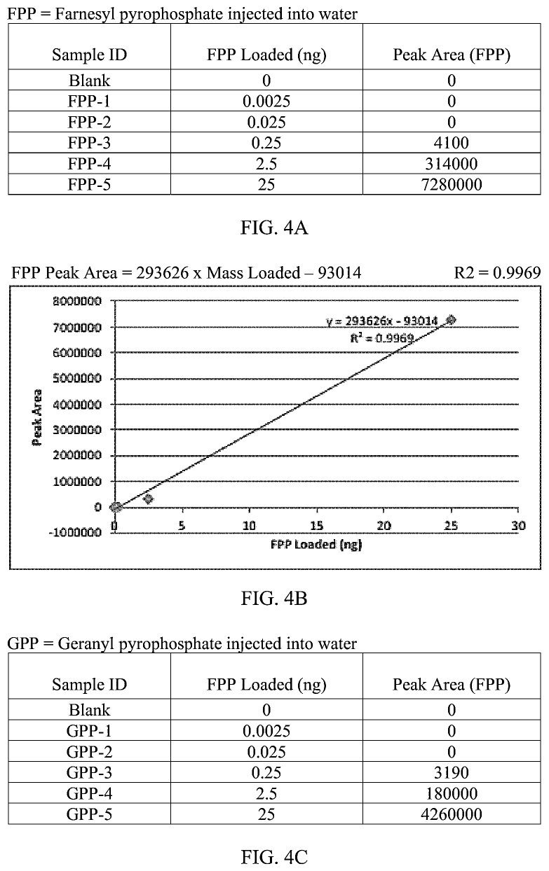

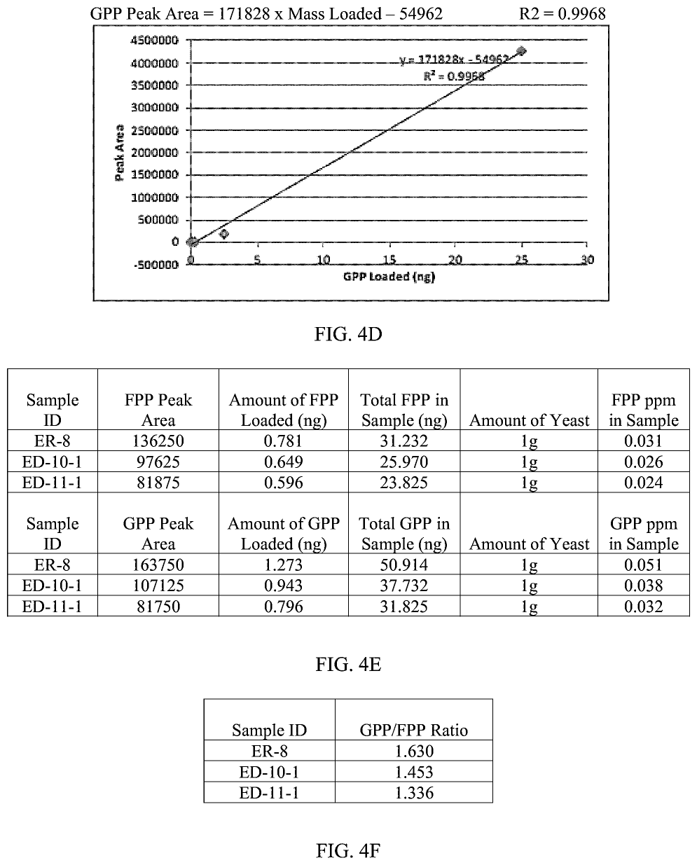

FIG. 4 depicts LC-MS/MS analysis. Panel A depicts Farnesyl pyrophosphate injected into water. Panel B is a graph showing Peak Area versus FPP Loaded. Panel C depicts Geranyl pyrophosphates injected into water. Panel D is a graph showing Peak Area versus GPP Loaded. Panel E shows a summary of the data. Panel F shows the ratio of GPP/FPP.

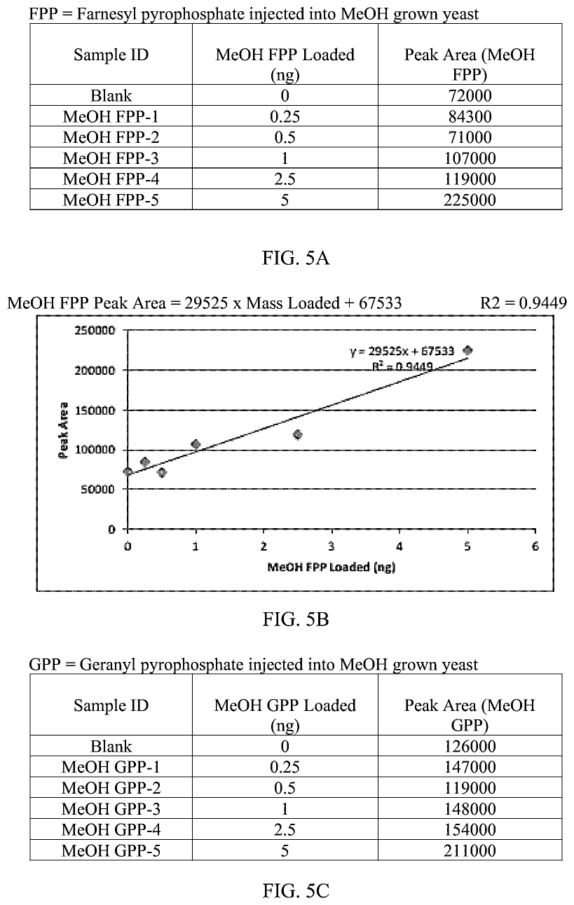

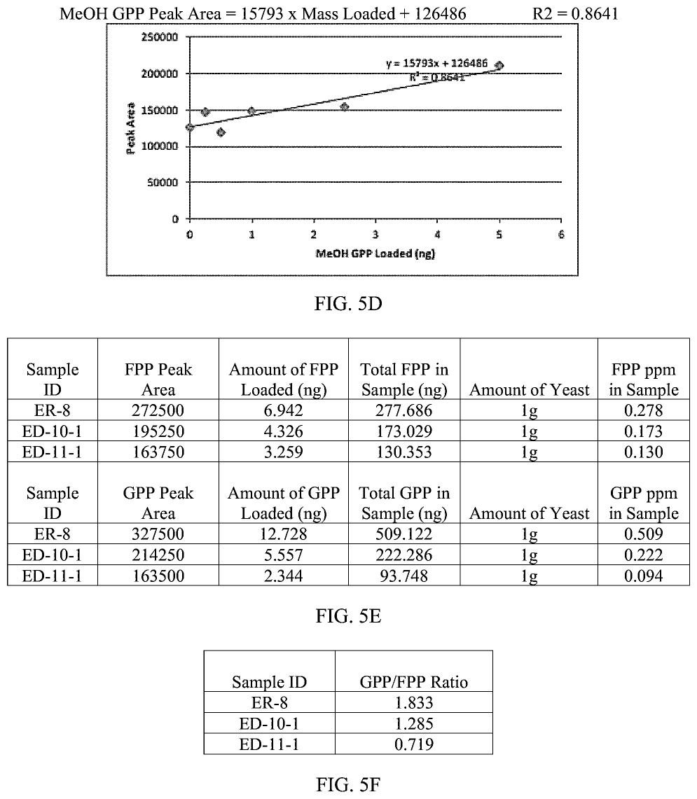

FIG. 5 depicts LC-MS/MS analysis. Panel A depicts Farnesyl pyrophosphate injected into MeOH grown yeast. Panel B is a graph showing Peak Area versus MeOH FPP Loaded. Panel C depicts Geranyl pyrophosphates injected into MeOH grown in yeast. Panel D is a graph showing Peak Area versus MeOH GPP Loaded. Panel E shows a summary of the data. Panel F shows the ratio of GPP/FPP.

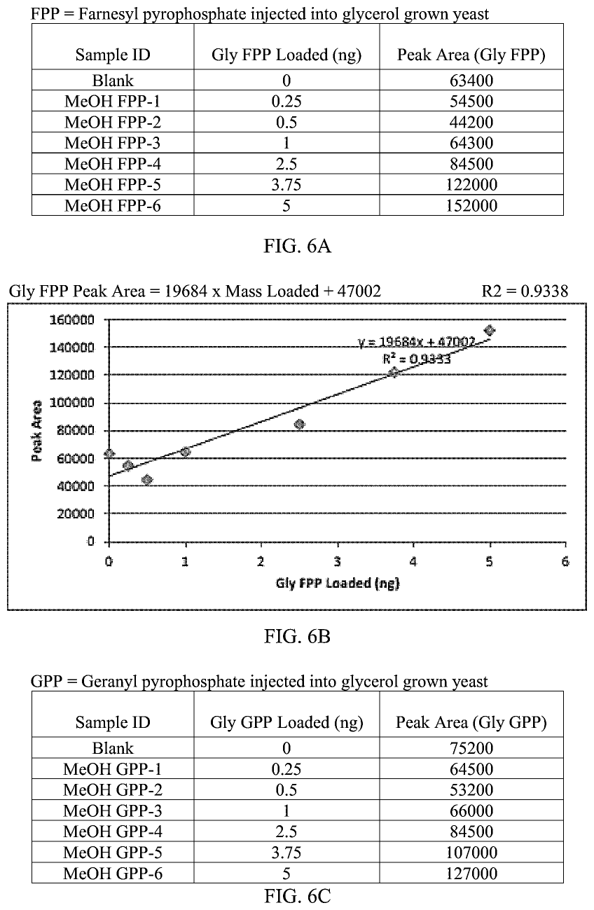

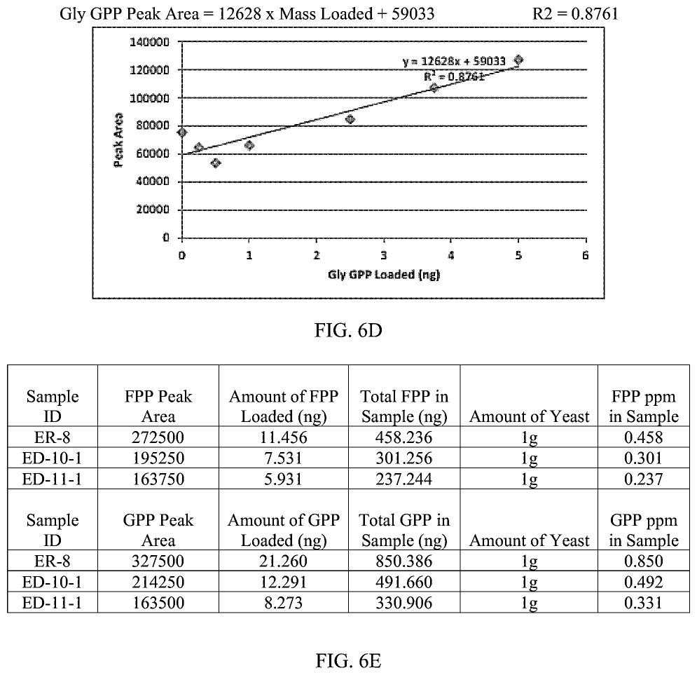

FIG. 6 depicts LC-MS/MS analysis. Panel A depicts Farnesyl pyrophosphate injected into glycerol grown yeast. Panel B is a graph showing Peak Area versus glycerol FPP Loaded. Panel C depicts Geranyl pyrophosphates injected into glycerol grown in yeast. Panel D is a graph showing Peak Area versus glycerol GPP Loaded. Panel E shows a summary of the data.

### Production of Cannabigerolic Acid

In one aspect, the present disclosure provides production of cannabigerolic acid (CBGA) in a methylotrophic yeast. In one example, the methylotrophic yeast is P. pastoris.

As described herein, cannabigerolic acid (CBGA) was produced in a methylotrophic yeast.

The inventors, through genetic engineering, have introduced the pathway required for the conversion of hexanoyl-CoA to olevitolic acid. This pathway begins by a heterologous polyketide synthase (CsTKS) combining one molecule of hexanoyl-CoA with three molecules of malonoyl-CoA to generate 3,5,7-trioxododecanoyl-CoA. 3,5,7-trioxododecanoyl-CoA is cyclized through the activity of an olevitolic acid cyclase (CsOAC) enzyme releasing olevitolic acid and a molecule of coenzyme A. An aromatic prenyltransferase (CsPT4) catalyzes the final step in the reaction pathway combining the olevitolic acid produced in the previous steps with GPP resulting in the production of CBGA.

In one example, CsTKS, CsOAC, and CsPT4, are from Cannabis sativa.

Thus, in one aspect, there is described herein a metabolic engineering strategy in which a CBGA was produced in P. pastoris.

In one example, there is provided a method of producing cannabigerolic acid (CBGA), in a methylotrophic yeast host cell, comprising: introducing a first polynucleotide encoding olevitiolic synthase polypeptide, introducing a second polynucleotide encoding olevitiolic acid cyclase polypeptide, and introducing a third polynucleotide encoding aromatic prenyl transferase, and culturing the methylotrophic yeast host cell under conditions sufficient for CBGA production.

In one example, there is provided a method of producing cannabigerolic acid (CBGA), in a methylotrophic yeast host cell, comprising: introducing a fourth polynucleotide encoding olevitiolic synthase polypeptide and encoding olevitiolic acid cyclase polypeptide, and introducing a third polynucleotide encoding aromatic prenyl transferase, culturing the methylotrophic yeast host cell under conditions sufficient for CBGA production.

In one example, there is provided a method of producing cannabigerolic acid (CBGA), in a methylotrophic yeast host cell, comprising: introducing a first polynucleotide encoding olevitiolic synthase polypeptide, and introducing a fifth polynucleotide encoding olevitiolic acid cyclase polypeptide and encoding acormatic prenyl transferase, culturing the methylotrophic yeast host cell under conditions sufficient for CBGA production.

In one example, the host cell is from the Komagataella genus.

In one example, the methylotrophic yeast host cell is Pichia pastoris (Komagataella phaffi).

In one example, the CsPT4/CsOAC sequence is:

(SEQ ID NO: 3)
GATCTAACATCCAAAGACGAAAGGTTGAATGAAACCTTTTTGCCATCCGA
CATCCACAGGTCCATTCTCACACATAAGTGCCAAACGCAACAGGAGGGGA
TACACTAGCAGCAGACCGTTGCAAACGCAGGACCTCCACTCCTCTTCTCC
TCAACACCCACTTTTGCCATCGAAAAACCAGCCCAGTTATTGGGCTTGAT
TGGAGCTCGCTCATTCCAATTCCTTCTATTAGGCTACTAACACCATGACT
TTATTAGCCTGTCTATCCTGGCCCCCCTGGCGAGGTTCATGTTTGTTTAT
TTCCGAATGCAACAAGCTCCGCATTACACCCGAACATCACTCCAGATGAG
GGCTTTCTGAGTGTGGGGTCAAATAGTTTCATGTTCCCCAAATGGCCCAA
AACTGACAGTTTAAACGCTGTCTTGGAACCTAATATGACAAAAGCGTGAT
CTCATCCAAGATGAACTAAGTTTGGTTCGTTGAAATGCTAACGGCCAGTT
GGTCAAAAAGAAACTTCCAAAAGTCGGCATACCGTTTGTCTTGTTTGGTA
TTGATTGACGAATGCTCAAAAATAATCTCATTAATGCTTAGCGCAGTCTC
TCTATCGCTTCTGAACCCCGGTGCACCTGTGCCGAAACGCAAATGGGGAA
ACACCCGCTTTTTGGATGATTATGCATTGTCTCCACATTGTATGCTTCCA
AGATTCTGGTGGGAATACTGCTGATAGCCTAACGTTCATGATCAAAATTT
AACTGTTCTAACCCCTACTTGACAGCAATATATAAACAGAAGGAAGCTGC
CCTGTCTTAAACCTTTTTTTTTATCATCATTATTAGCTTACTTTCATAAT
TGCGACTGGTTCCAATTGACAAGCTTTTGATTTTAACGACTTTTAACGAC
AACTTGAGAAGATCAAAAAACAACTAATTATTCGAAACGATGCATCATCA
TCATCACCATTCCTCCGGTAGAGAGAACTTGTACTTCCAAGGTATGTCTG
CTGGTTCTGACCAAATCGAAGGTTCCCCACACCACGAGTCTGATAACTCC
ATTGCTACCAAGATTTTGAACTTTGGCCATACGTGTTGGAAATTGCAAAG
ACCTTACGTTGTCAAGGGTATGATATCGATTGCTTGTGGTTTGTTTGGTA
GAGAGTTGTTCAACAACAGACATTTGTTCTCTTGGGGTTTGATGTGGAAG
GCTTTTTTTGCCCTTGTTCCTATTCTTTCTTTCAACTTTTTTGCCGCTAT
CATGAACCAGATTTACGATGTTGATATTGACAGAATCAACAAGCCAGACC
TTCCATTGGTTTCTGGTGAGATGTCCATTGAGACTGCTTGGATTCTGTCA
ATTATAGTTGCTTTGACTGGTTTGATTGTCACTATCAAGCTGAAATCTGC
TCCACTGTTTGTTTTCATTTACATCTTTGGTATTTTTGCTGGTTTTGCCT
ATTCGGTTCCTCCAATTAGATGGAAGCAATATCCATTCACCAACTTTCTG
ATCACAATTTCCTCTCACGTTGGTTTGGCTTTCACAAGCTACTCCGCTAC
TACCTCTGCTTTGGGTCTTCCATTCGTGTGGCGTCCAGCCTTTTCTTTCA
TCATTGCTTTCATGACTGTCATGGGTATGACCATTGCTTTTGCCAAGGAC
ATTTCTGATATCGAAGGTGACGCTAAGTACGGTGTCTCGACCGTTGCTAC
CAAGTTGGGTGCCAGAAATATGACTTTCGTTGTTTCTGGTGTTTTGCTGT
TGAACTACCTGGTGAGTATTTCAATTGGTATTATCTGGCCCCAAGTCTTC
AAGTCCAACATTATGATTCTATCTCACGCTATTTTGGCTTTCTGTTTGAT
CTTCCAAACCAGAGAGCTGGCTTTGGCCAACTACGCTTCCGCTCCATCCA
GACAATTCTTTGAATTCATTTGGCTATTGTACTACGCCGAGTACTTCGTT
TATGTTTTCATCTAATGAAATGTATTTAATTTGATATTAAGTAAATGAAT
GATTATGACTTTATGAATTCGCAATGTTTTCTCCTTGATTATTTCTGTAT
TGTATTGGAATGATTATAGAATACTCATATATTGATTATAGTATTAGCAC
ATAAAACGTTTGTTGTTAAACTCACTTCCGTACGCAACCATTTCTATTTC
TAGCTATCTTGATAAGGTGATCTAACATCCAAAGACGAAAGGTTGAATGA
AACCTTTTTGCCATCCGACATCCACAGGTCCATTCTCACACATAAGTGCC
AAACGCAACAGGAGGGGATACACTAGCAGCAGACCGTTGCAAACGCAGGA
CCTCCACTCCTCTTCTCCTCAACACCCACTTTTGCCATCGAAAAACCAGC
CCAGTTATTGGGCTTGATTGGAGCTCGCTCATTCCAATTCCTTCTATTAG
GCTACTAACACCATGACTTTATTAGCCTGTCTATCCTGGCCCCCCTGGCG
AGGTTCATGTTTGTTTATTTCCGAATGCAACAAGCTCCGCATTACACCCG
AACATCACTCCAGATGAGGGCTTTCTGAGTGTGGGGTCAAATAGTTTCAT
GTTCCCCAAATGGCCCAAAACTGACAGTTTAGAGACCTATTCCAGACTTC
AGATCTCCAAAAACAGGACTATATCATAATTTGGCACGTTTGAATCTTCC
CCATCCTGAAGCGGTATTTGAGATCAACTACTTCAGAGAAAACCCTCATG
CTTTTTACACATTGGCTGACGAACTTTACCCTGGGAGGGTCTCAAAACGC
TGTCTTGGAACCTAATATGACAAAAGCGTGATCTCATCCAAGATGAACTA
AGTTTGGTTCGTTGAAATGCTAACGGCCAGTTGGTCAAAAAGAAACTTCC
AAAAGTCGGCATACCGTTTGTCTTGTTTGGTATTGATTGACGAATGCTCA
AAAATAATCTCATTAATGCTTAGCGCAGTCTCTCTATCGCTTCTGAACCC
CGGTGCACCTGTGCCGAAACGCAAATGGGGAAACACCCGCTTTTTGGATG
ATTATGCATTGTCTCCACATTGTATGCTTCCAAGATTCTGGTGGGAATAC
TGCTGATAGCCTAACGTTCATGATCAAAATTTAACTGTTCTAACCCCTAC
TTGACAGCAATATATAAACAGAAGGAAGCTGCCCTGTCTTAAACCTTTTT
TTTTATCATCATTATTAGCTTACTTTCATAATTGCGACTGGTTCCAATTG
ACAAGCTTTTGATTTTAACGACTTTTAACGACAACTTGAGAAGATCAAAA
AACAACTAATTATTCGAAACGATGCATCATCATCATCATCATAGCTCCGG
TAGAGAGAACTTGTACTTCCAAGGTATGGCCGTTAAGCATCTGATCGTTT
TAAAGTTCAAGGATGAGATCACCGAGGCTCAAAAGGAAGAGTTTTTCAAG
ACTTACGTCAACTTGGTCAACATCATTCCAGCTATGAAGGATGTCTACTG
GGGTAAGGACGTCACTCAAAAGAACAAGGAGGAGGGATACACCCACATAG
TGGAGGTTACTTTCGAGTCTGTCGAGACTATCCAGGACTACATCATTCAC
CCAGCCCACGTTGGATTCGGCGATGTCTACAGATCTTTTTGGGAGAAGTT
GCTGATCTTTGACTACACTCCAAGAAAAGGTTCATAATCAAGAGGATGTC
AGAATGCCATTTGCCTGAGAGATGCAGGCTTCATTTTTGATACTTTTTTA
TTTGTAACCTATATAGTATAGGATTTTTTTTGTCATTTTGTTTCTTCTCG
TACGAGCTTGCTCCTGATCAGCCTATCTCGCAGCTGATGAATATCTTGTG
GTAGGGGTTTGGGAAAATCATTCGAGTTTGATGTTTTTCTTGGTATTTCC
CACTCCTCTTCAGAGTACAGAAGATTAAGTGACACGTTCGTTTGTGCAAG
CTTCAACGATGCCAAAAGGGTATAATAAGCGTCATTTGCAGCATTGTGAA
GAAAACTATGTGGCAAGCCAAGCCTGCGAAGAATGTA.

In one example, the CsPT4 polypeptide sequence is:

(SEQ ID NO: 4)
MHHHHHHSSGRENLYFQGMSAGSDQIEGSPHHESDNSIATKILNFGHTCW
KLQRPYVVKGMISIACGLFGRELFNNRHLFSWGLMWKAFFALVPILSFNF
FAAIMNQIYDVDIDRINKPDLPLVSGEMSIETAWILSIIVALTGLIVTIK
LKSAPLFVFIYIFGIFAGFAYSVPPIRWKQYPFTNFLITISSHVGLAFTS
YSATTSALGLPFVWRPAFSFIIAFMTVMGMTIAFAKDISDIEGDAKYGVS
TVATKLGARNMTFVVSGVLLLNYLVSISIGIIWPQVFKSNIMILSHAILA
FCLIFQTRELALANYASAPSRQFFEFIWLLYYAEYFVYVFI

In one example, the CsOAC polypeptide sequence is:

(SEQ ID NO: 5)
MHHHHHHSSGRENLYFQGMAVKHLIVLKFKDEITEAQKEEFFKTYVNLVN
IIPAMKDVYWGKDVTQKNKEEGYTHIVEVTFESVETIQDYIIHPAHVGFG
DVYRSFWEKLLIFDYTPRKGS

In one example, the CsTKS polynucleotide sequence is:

(SEQ ID NO: 6)
ATGCATCATCATCATCATCATTCCTCCGGTAGAGAGAACTTGTACTTCCA
AGGTATGAACCATTTGAGAGCTGAAGGTCCTGCTTCCGTTTTGGCTATTG
GAACTGCCAATCCAGAGAACATCCTTTTGCAAGATGAGTTTCCAGACTAC
TATTTCCGTGTCACCAAGTCTGAACACATGACTCAATTGAAGGAGAAGTT
CAGAAAGATTTGTGATAAGTCAATGATCAGAAAGAGAAACTGTTTTTTGA
ACGAGGAACATTTGAAACAAAACCCAAGATTAGTTGAGCATGAAATGCAA
ACATTGGACGCTAGACAGGATATGTTGGTTGTTGAGGTTCCAAAGTTGGG
TAAGGATGCTTGTGCCAAGGCCATCAAGGAATGGGGTCAGCCAAAGTCCA
AGATTACTCACTTGATCTTTACAAGCGCTTCTACCACTGACATGCCAGGA
GCCGACTACCACTGCGCCAAGTTGTTGGGACTGTCTCCTTCTGTTAAGAG
AGTTATGATGTACCAATTGGGATGTTACGGTGGTGGTACCGTTTTGAGAA
TTGCCAAGGACATTGCTGAGAACAACAAGGGTGCCAGAGTTTTGGCTGTT
TGTTGTGATATCATGGCTTGTTTGTTCAGAGGACCAAGCGAATCAGATTT
GGAATTGTTGGTTGGCCAAGCCATTTTTGGAGATGGTGCTGCTGCTGTTA
TTGTTGGTGCCGAACCAGACGAATCTGTTGGAGAGAGACCAATCTTCGAA
TTAGTTTCGACTGGTCAGACAATTTTGCCCAACTCTGAAGGAACAATTGG
TGGTCACATCAGAGAAGCTGGTTTGATCTTCGACTTGCACAAGGATGTAC
CAATGCTGATTTCCAACAACATTGAGAAGTGTTTGATTGAGGCTTTTACC
CCTATTGGTATTTCTGATTGGAACTCCATCTTCTGGATCACTCATCCAGG
TGGTAAGGCCATTTTGGACAAAGTTGAGGAGAAGTTGCACTTGAAGTCTG
ATAAGTTTGTTGACTCCAGACACGTGCTCTCTGAGCACGGTAATATGTCA
TCTTCCACTGTTCTGTTCGTCATGGATGAGTTGAGAAAGAGATCTTTGGA
GGAAGGTAAGTCTACTACAGGCGATGGATTTGAATGGGGTGTTTTGTTCG
GTTTCGGGCCAGGTTTGACCGTCGAGAGAGTTGTTGTTAGATCTGTTCCA
ATTAAGTACGGATCTTAA.

In one example, the CsTKS polypeptide sequence is:

(SEQ ID NO: 7)
MHHHHHHSSGRENLYFQGMNHLRAEGPASVLAIGTANPENILLQDEFPDY
YFRVTKSEHMTQLKEKFRKICDKSMIRKRNCFLNEEHLKQNPRLVEHEMQ
TLDARQDMLVVEVPKLGKDACAKAIKEWGQPKSKITHLIFTSASTTDMPG
ADYHCAKLLGLSPSVKRVMMYQLGCYGGGTVLRIAKDIAENNKGARVLAV
CCDIMACLFRGPSESDLELLVGQAIFGDGAAAVIVGAEPDESVGERPIFE
LVSTGQTILPNSEGTIGGHIREAGLIFDLHKDVPMLISNNIEKCLIEAFT
PIGISDWNSIFWITHPGGKAILDKVEEKLHLKSDKFVDSRHVLSEHGNMS
SSTVLFVMDELRKRSLEEGKSTTGDGFEWGVLFGFGPGLTVERVVVRSVP
IKYGS.

In one example, the CsPT4 polynucleotide sequence is:

(SEQ ID NO: 8)
ATGCATCATCATCATCACCATTCCTCCGGTAGAGAGAACTTGTACTTCCA
AGGTATGTCTGCTGGTTCTGACCAAATCGAAGGTTCCCCACACCACGAGT
CTGATAACTCCATTGCTACCAAGATTTTGAACTTTGGCCATACGTGTTGG
AAATTGCAAAGACCTTACGTTGTCAAGGGTATGATATCGATTGCTTGTGG
TTTGTTTGGTAGAGAGTTGTTCAACAACAGACATTTGTTCTCTTGGGGTT
TGATGTGGAAGGCTTTTTTTGCCCTTGTTCCTATTCTTTCTTTCAACTTT
TTTGCCGCTATCATGAACCAGATTTACGATGTTGATATTGACAGAATCAA
CAAGCCAGACCTTCCATTGGTTTCTGGTGAGATGTCCATTGAGACTGCTT
GGATTCTGTCAATTATAGTTGCTTTGACTGGTTTGATTGTCACTATCAAG
CTGAAATCTGCTCCACTGTTTGTTTTCATTTACATCTTTGGTATTTTTGC
TGGTTTTGCCTATTCGGTTCCTCCAATTAGATGGAAGCAATATCCATTCA
CCAACTTTCTGATCACAATTTCCTCTCACGTTGGTTTGGCTTTCACAAGC
TACTCCGCTACTACCTCTGCTTTGGGTCTTCCATTCGTGTGGCGTCCAGC
CTTTTCTTTCATCATTGCTTTCATGACTGTCATGGGTATGACCATTGCTT
TTGCCAAGGACATTTCTGATATCGAAGGTGACGCTAAGTACGGTGTCTCG
ACCGTTGCTACCAAGTTGGGTGCCAGAAATATGACTTTCGTTGTTTCTGG
TGTTTTGCTGTTGAACTACCTGGTGAGTATTTCAATTGGTATTATCTGGC
CCCAAGTCTTCAAGTCCAACATTATGATTCTATCTCACGCTATTTTGGCT
TTCTGTTTGATCTTCCAAACCAGAGAGCTGGCTTTGGCCAACTACGCTTC
CGCTCCATCCAGACAATTCTTTGAATTCATTTGGCTATTGTACTACGCCG
AGTACTTCGTTTATGTTTTCATCTAA.

In one example, the CsOAC polynucleotide sequence is:

(SEQ ID NO: 9)
ATGCATCATCATCATCATCATAGCTCCGGTAGAGAGAACTTGTACTTCCA
AGGTATGGCCGTTAAGCATCTGATCGTTTTAAAGTTCAAGGATGAGATCA
CCGAGGCTCAAAAGGAAGAGTTTTTCAAGACTTACGTCAACTTGGTCAAC
ATCATTCCAGCTATGAAGGATGTCTACTGGGGTAAGGACGTCACTCAAAA
GAACAAGGAGGAGGGATACACCCACATAGTGGAGGTTACTTTCGAGTCTG
TCGAGACTATCCAGGACTACATCATTCACCCAGCCCACGTTGGATTCGGC
GATGTCTACAGATCTTTTTGGGAGAAGTTGCTGATCTTTGACTACACTCC
AAGAAAAGGTTCATAA

In one example, the ScPT4/CsOAC polynucleotide is:

(SEQ ID NO: 10)
GATCTAACATCCAAAGACGAAAGGTTGAATGAAACCTTTTTGCCATCCGA
CATCCACAGGTCCATTCTCACACATAAGTGCCAAACGCAACAGGAGGGGA
TACACTAGCAGCAGACCGTTGCAAACGCAGGACCTCCACTCCTCTTCTCC
TCAACACCCACTTTTGCCATCGAAAAACCAGCCCAGTTATTGGGCTTGAT
TGGAGCTCGCTCATTCCAATTCCTTCTATTAGGCTACTAACACCATGACT
TTATTAGCCTGTCTATCCTGGCCCCCCTGGCGAGGTTCATGTTTGTTTAT
TTCCGAATGCAACAAGCTCCGCATTACACCCGAACATCACTCCAGATGAG
GGCTTTCTGAGTGTGGGGTCAAATAGTTTCATGTTCCCCAAATGGCCCAA
AACTGACAGTTTAAACGCTGTCTTGGAACCTAATATGACAAAAGCGTGAT
CTCATCCAAGATGAACTAAGTTTGGTTCGTTGAAATGCTAACGGCCAGTT
GGTCAAAAAGAAACTTCCAAAAGTCGGCATACCGTTTGTCTTGTTTGGTA
TTGATTGACGAATGCTCAAAAATAATCTCATTAATGCTTAGCGCAGTCTC
TCTATCGCTTCTGAACCCCGGTGCACCTGTGCCGAAACGCAAATGGGGAA
ACACCCGCTTTTTGGATGATTATGCATTGTCTCCACATTGTATGCTTCCA
AGATTCTGGTGGGAATACTGCTGATAGCCTAACGTTCATGATCAAAATTT
AACTGTTCTAACCCCTACTTGACAGCAATATATAAACAGAAGGAAGCTGC
CCTGTCTTAAACCTTTTTTTTTATCATCATTATTAGCTTACTTTCATAAT
TGCGACTGGTTCCAATTGACAAGCTTTTGATTTTAACGACTTTTAACGAC
AACTTGAGAAGATCAAAAAACAACTAATTATTCGAAACGATGCATCATCA
TCATCACCATTCCTCCGGTAGAGAGAACTTGTACTTCCAAGGTATGTCTG
CTGGTTCTGACCAAATCGAAGGTTCCCCACACCACGAGTCTGATAACTCC
ATTGCTACCAAGATTTTGAACTTTGGCCATACGTGTTGGAAATTGCAAAG
ACCTTACGTTGTCAAGGGTATGATATCGATTGCTTGTGGTTTGTTTGGTA
GAGAGTTGTTCAACAACAGACATTTGTTCTCTTGGGGTTTGATGTGGAAG
GCTTTTTTTGCCCTTGTTCCTATTCTTTCTTTCAACTTTTTTGCCGCTAT
CATGAACCAGATTTACGATGTTGATATTGACAGAATCAACAAGCCAGACC
TTCCATTGGTTTCTGGTGAGATGTCCATTGAGACTGCTTGGATTCTGTCA
ATTATAGTTGCTTTGACTGGTTTGATTGTCACTATCAAGCTGAAATCTGC
TCCACTGTTTGTTTTCATTTACATCTTTGGTATTTTTGCTGGTTTTGCCT
ATTCGGTTCCTCCAATTAGATGGAAGCAATATCCATTCACCAACTTTCTG
ATCACAATTTCCTCTCACGTTGGTTTGGCTTTCACAAGCTACTCCGCTAC
TACCTCTGCTTTGGGTCTTCCATTCGTGTGGCGTCCAGCCTTTTCTTTCA
TCATTGCTTTCATGACTGTCATGGGTATGACCATTGCTTTTGCCAAGGAC
ATTTCTGATATCGAAGGTGACGCTAAGTACGGTGTCTCGACCGTTGCTAC
CAAGTTGGGTGCCAGAAATATGACTTTCGTTGTTTCTGGTGTTTTGCTGT
TGAACTACCTGGTGAGTATTTCAATTGGTATTATCTGGCCCCAAGTCTTC
AAGTCCAACATTATGATTCTATCTCACGCTATTTTGGCTTTCTGTTTGAT
CTTCCAAACCAGAGAGCTGGCTTTGGCCAACTACGCTTCCGCTCCATCCA
GACAATTCTTTGAATTCATTTGGCTATTGTACTACGCCGAGTACTTCGTT
TATGTTTTCATCTAATGAAATGTATTTAATTTGATATTAAGTAAATGAAT
GATTATGACTTTATGAATTCGCAATGTTTTCTCCTTGATTATTTCTGTAT
TGTATTGGAATGATTATAGAATACTCATATATTGATTATAGTATTAGCAC
ATAAAACGTTTGTTGTTAAACTCACTTCCGTACGCAACCATTTCTATTTC
TAGCTATCTTGATAAGGTGATCTAACATCCAAAGACGAAAGGTTGAATGA
AACCTTTTTGCCATCCGACATCCACAGGTCCATTCTCACACATAAGTGCC
AAACGCAACAGGAGGGGATACACTAGCAGCAGACCGTTGCAAACGCAGGA
CCTCCACTCCTCTTCTCCTCAACACCCACTTTTGCCATCGAAAAACCAGC
CCAGTTATTGGGCTTGATTGGAGCTCGCTCATTCCAATTCCTTCTATTAG
GCTACTAACACCATGACTTTATTAGCCTGTCTATCCTGGCCCCCCTGGCG
AGGTTCATGTTTGTTTATTTCCGAATGCAACAAGCTCCGCATTACACCCG
AACATCACTCCAGATGAGGGCTTTCTGAGTGTGGGGTCAAATAGTTTCAT
GTTCCCCAAATGGCCCAAAACTGACAGTTTAGAGACCTATTCCAGACTTC
AGATCTCCAAAAACAGGACTATATCATAATTTGGCACGTTTGAATCTTCC
CCATCCTGAAGCGGTATTTGAGATCAACTACTTCAGAGAAAACCCTCATG
CTTTTTACACATTGGCTGACGAACTTTACCCTGGGAGGGTCTCAAAACGC
TGTCTTGGAACCTAATATGACAAAAGCGTGATCTCATCCAAGATGAACTA
AGTTTGGTTCGTTGAAATGCTAACGGCCAGTTGGTCAAAAAGAAACTTCC
AAAAGTCGGCATACCGTTTGTCTTGTTTGGTATTGATTGACGAATGCTCA
AAAATAATCTCATTAATGCTTAGCGCAGTCTCTCTATCGCTTCTGAACCC
CGGTGCACCTGTGCCGAAACGCAAATGGGGAAACACCCGCTTTTTGGATG
ATTATGCATTGTCTCCACATTGTATGCTTCCAAGATTCTGGTGGGAATAC
TGCTGATAGCCTAACGTTCATGATCAAAATTTAACTGTTCTAACCCCTAC
TTGACAGCAATATATAAACAGAAGGAAGCTGCCCTGTCTTAAACCTTTTT
TTTTATCATCATTATTAGCTTACTTTCATAATTGCGACTGGTTCCAATTG
ACAAGCTTTTGATTTTAACGACTTTTAACGACAACTTGAGAAGATCAAAA
AACAACTAATTATTCGAAACGATGCATCATCATCATCATCATAGCTCCGG
TAGAGAGAACTTGTACTTCCAAGGTATGGCCGTTAAGCATCTGATCGTTT
TAAAGTTCAAGGATGAGATCACCGAGGCTCAAAAGGAAGAGTTTTTCAAG
ACTTACGTCAACTTGGTCAACATCATTCCAGCTATGAAGGATGTCTACTG
GGGTAAGGACGTCACTCAAAAGAACAAGGAGGAGGGATACACCCACATAG
TGGAGGTTACTTTCGAGTCTGTCGAGACTATCCAGGACTACATCATTCAC
CCAGCCCACGTTGGATTCGGCGATGTCTACAGATCTTTTTGGGAGAAGTT
GCTGATCTTTGACTACACTCCAAGAAAAGGTTCATAATCAAGAGGATGTC
AGAATGCCATTTGCCTGAGAGATGCAGGCTTCATTTTTGATACTTTTTTA
TTTGTAACCTATATAGTATAGGATTTTTTTTGTCATTTTGTTTCTTCTCG
TACGAGCTTGCTCCTGATCAGCCTATCTCGCAGCTGATGAATATCTTGTG
GTAGGGGTTTGGGAAAATCATTCGAGTTTGATGTTTTTCTTGGTATTTCC
CACTCCTCTTCAGAGTACAGAAGATTAAGTGACACGTTCGTTTGTGCAAG
CTTCAACGATGCCAAAAGGGTATAATAAGCGTCATTTGCAGCATTGTGAA
GAAAACTATGTGGCAAGCCAAGCCTGCGAAGAATGTAGTCGAGAATTGAG
CTTGC

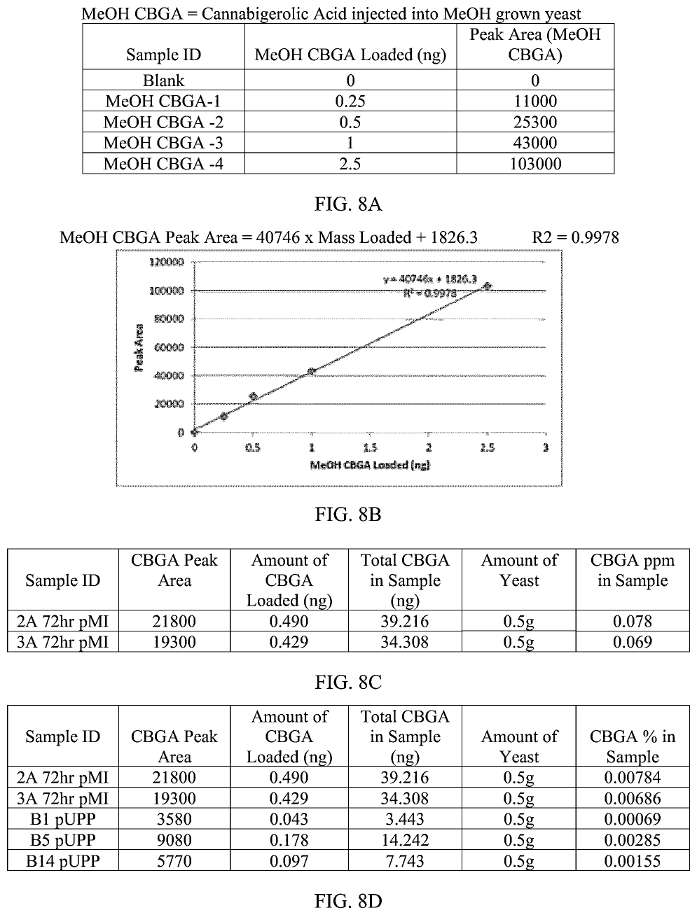

FIG. 8 depicts Quantification of MeOH induced P. pastoris CBGA levels. Calibration curve and analysis of LC/MS data showing the quantification of the CBGA produced in P. pastoris. Panel A depicts CBGA injected into MeOH grown yeast. Panel B should a graph of Peak Area versus MeOH CBGA. Panel C shows a summary of data. Panel D shows a summary of the data.

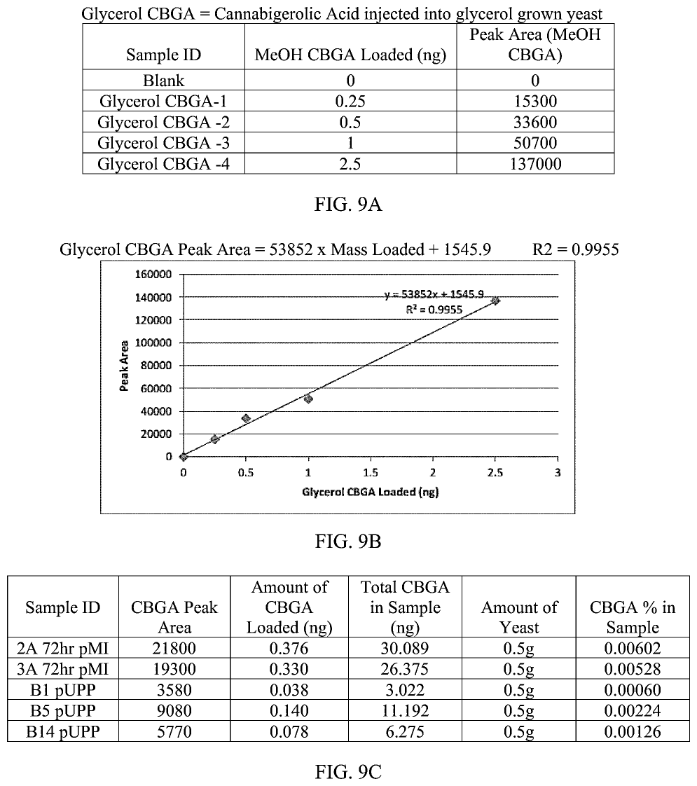

FIG. 9 depicts

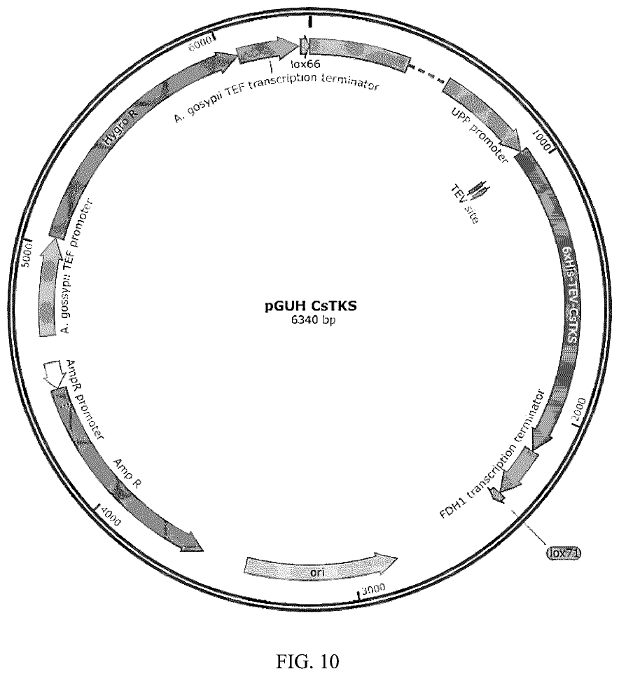

FIG. 10 depicts the pGUH CsTKS map.

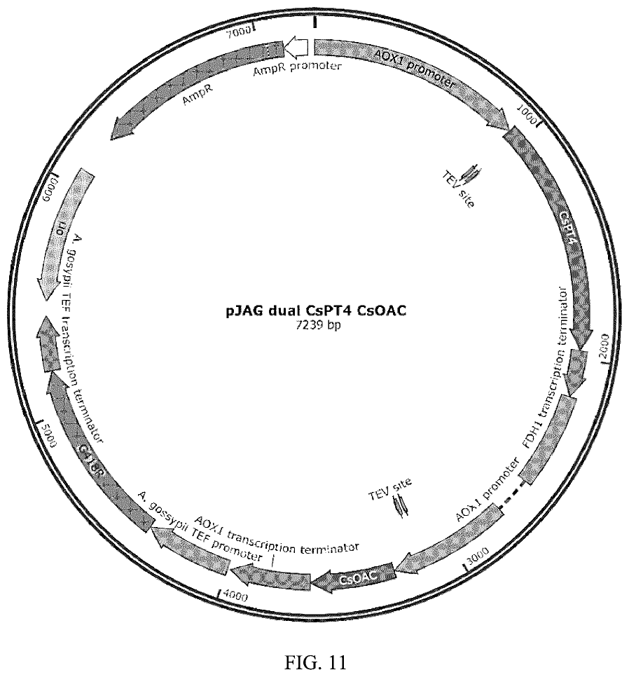

FIG. 11 depicts the pJAG dual CsPT4 CsOAC map.

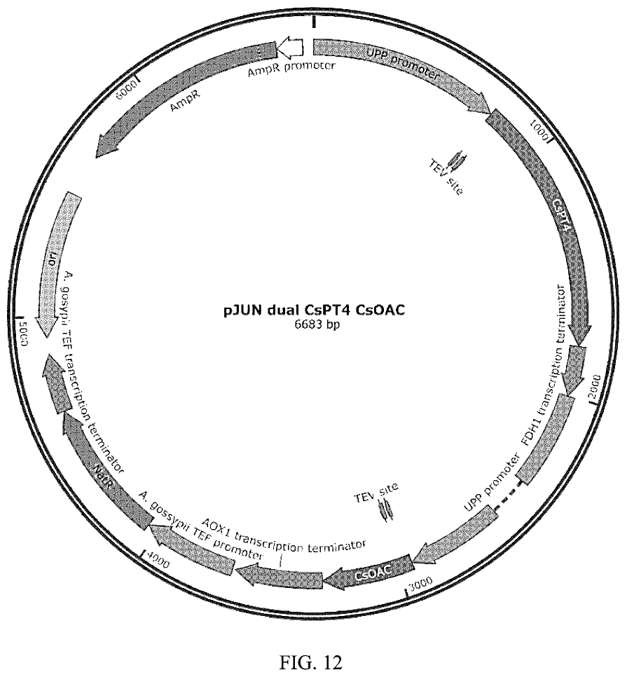

FIG. 12 depicts the pJUN dual CsPT4 CsOAC map.

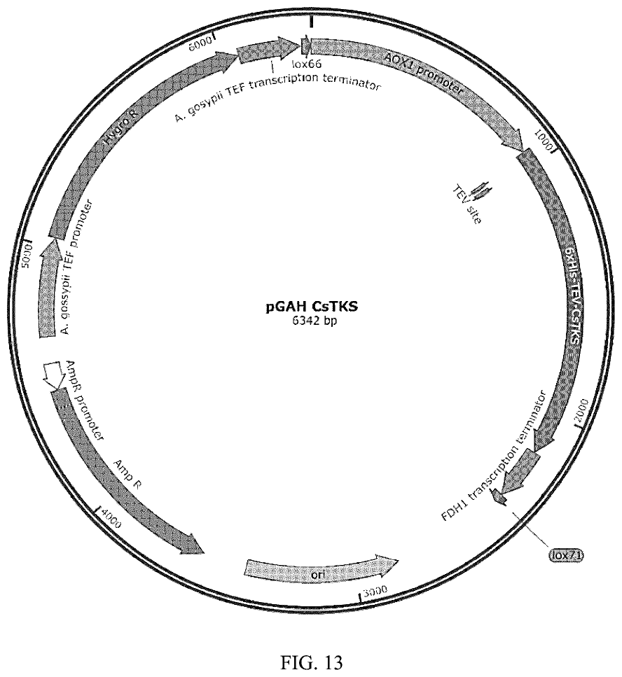

FIG. 13 depicts the pGAH CsTKS map.

In one example, CBGA production was introducing CsTKS, CsOAC, and CsPT4, in to the methylotropic yeast, P. pastoris.

As used herein, the term “polynucleotide(s)”, “nucleic acid molecule(s)” or “nucleic acid sequence(s)” are interchangeable and it generally refers to any polyribonucleotide or poly-deoxyribonucleotide, which may be unmodified RNA or DNA or modified RNA or DNA or any combination thereof. As used herein, the term “nucleic acid(s)” also includes DNAs or RNAs as described above that contain one or more modified bases. Thus, DNAs or RNAs with backbones modified for stability or for other reasons are “nucleic acids”. The term “nucleic acids” as it is used herein embraces such chemically, enzymatically or metabolically modified forms of nucleic acids, as well as the chemical forms of DNA and RNA characteristic of viruses and cells, including for example, simple and complex cells. In some examples, one or more nucleotides are modified at the base, sugar or backbone to make the nucleic acid more stable or less likely to be cleared e.g. phosphorothioate backbone, pegylated backbone.

A polynucleotide may consist of an entire gene, or any portion thereof.

A gene is a polynucleotide that encodes (which may also be referred to as “coding for”) a functional polypeptide or RNA molecule.

In some examples, polynucleotides as described herein may also encompass polynucleotide sequences that differ from the disclosed sequences (which may also be know as the reference sequence) but which, as a result of the degeneracy of genetic code, encode a polypeptide which is the same as that encoded by a polynucleotide of the present disclosure.

In some examples, the polynucleotides described herein are isolated and purified, as those terms are commonly used in the art.

The term “isolated” refers to sequences that are removed from their natural cellular or other naturally occurring biological environment or from the environment of the experiment. An isolated molecule may be obtained by any method or combination of methods including biochemical, recombinant, and synthetic techniques. The polypeptide sequences may be prepared by at least one purification step.

The nucleotide sequences of described herein, may be modified. In some examples, the polynucleotides contains one or more chemical modifications. The modifications may be various distinct modifications. In some embodiments, the regions may contain one, two, or more (optionally different) nucleoside or nucleotide modifications.

Polypeptides described herein may be produced by inserting a polynucleotide that encodes the polypeptide into an expression vector and expressing the polypeptide in an appropriate host.

Any of a variety of expression vectors known to those of ordinary skill in the art may be employed. Expression may be achieved in any appropriate host cell that has been transformed or transfected with an expression vector containing a polypeptide encoding a recombinant polypeptide.

The polynucleotide(s) described herein may be used in a vector.

The term “vector” as used herein refers to any nucleic acid molecule for the cloning of and/or transfer of a nucleic acid into a cell. A vector may be a replicon to which another nucleotide sequence may be attached to allow for replication of the attached nucleotide sequence.

A “replicon” can be any genetic element (for example a plasmid, phage, cosmid, chromosome, viral genome) that functions as an autonomous unit of nucleic acid replication in vivo, and for example, is capable of replication under its own control. The term “vector” includes both viral and non viral (e.g., plasmid) nucleic acid molecules for introducing a nucleic acid into a cell in vitro, ex vivo, and/or in vivo. A large number of vectors known in the art may be used to manipulate nucleic acids, incorporate response elements and promoters into genes, and the like. For example, the insertion of the nucleic acid fragments corresponding to response elements and promoters into a suitable vector can be accomplished by ligating the appropriate nucleic acid fragments into a chosen vector that has complementary cohesive termini. Alternatively, the ends of the nucleic acid molecules may be enzymatically modified or any site may be produced by ligating nucleotide sequences (linkers) to the nucleic acid termini. Such vectors may be engineered to contain sequences encoding selectable markers that provide for the selection of cells that contain the vector and/or have incorporated the nucleic acid of the vector into the cellular genome. Such markers allow identification and/or selection of host cells that incorporate and express the proteins encoded by the marker. A “recombinant” vector refers to a viral or non-viral vector that comprises one or more heterologous nucleotide sequences.

As used herein, the term “isolated” refers to material, for example a polynucleotide, a polypeptide, or a cell, that is substantially or essentially free from components that normally accompany it in its native state.

As used herein, the term “variant” refers to a polynucleotide or polypeptide sequences different from the specifically identified sequences, wherein one or more nucleotides or amino acid residues is deleted, substituted, or added. Variants may be naturally occurring allelic variants, or non-naturally occurring variants. Variant polynucleotide sequences preferably exhibit at least 70%; more preferably at least 80%; more preferably yet at least 90%; and most preferably at least 95% identity to a sequence of the present invention. Variant polypeptide sequences preferably exhibit at least 70%; more preferably at least 80%; more preferably yet at least 90%; and most preferably at least 95% identity to a sequences described herein.

In some examples, variant polynucleotides of the polynucleotides described herein hybridize to the polynucleotide sequences recited in SEQ ID NO:2, or complements, reverse sequences, or reverse complements of those sequences under high stringent conditions (also referred to as high stringency).

In one example, as used herein, “stringent conditions” refers to prewashing in a solution of 6×SSC, 0.2% SDS; hybridizing at 65 C, 6×SSC, 0.2% SDS overnight; followed by two washes of 30 minutes each in 1×SSC, 0.1% SDS at 65 C and two washes of 30 minutes each in 0.2×SSC, 0.1% SDS at 65 C.

The term “polypeptide” refers to amino acid chains of any length, including full length sequences in which amino acid residues are linked by covalent peptide bonds. Polypeptides may be isolated natural products, or may be produced partially or wholly using recombinant or synthetic techniques. Thus, the term “polypeptide” may also refer to “protein”.

The vector comprising a polynucleotide which encodes a fusion polypeptide may be introduced in to a cell.

The term “introducing” as used herein in the context of a cell or organism refers to presenting the nucleic acid molecule to the organism and/or cell in such a manner that the nucleic acid molecule gains access to the interior of a cell. Where more than one nucleic acid molecule is to be introduced these nucleic acid molecules can be assembled as part of a single polynucleotide or nucleic acid construct, or as separate polynucleotide or nucleic acid constructs, and can be located on the same or different nucleic acid constructs. Accordingly, these polynucleotides can be introduced into cells in a single transformation event or in separate transformation events.

Thus, the term “transformation” as used herein refers to the introduction of a nucleic acid into a cell. Transformation of a cell may be stable or transient.

The term “transient transformation” as used herein in the context of a polynucleotide refers to a polynucleotide that may be introduced into the cell and does not integrate into the genome of the cell.

The term “stably introducing” or “stably introduced” as used herein in the context of a polynucleotide introduced into a cell refers to a polynucleotide that may be stably incorporated into the genome of the cell, and thus the cell is stably transformed with the polynucleotide.

As used herein, the terms “contacting” refers to a process by which, for example, a compound may be delivered to a cell. The compound may be administered in a number of ways, including, but not limited to, direct introduction into a cell (i.e., intracellularly) and/or extracellular introduction into a cavity, interstitial space, or into the circulation of the organism.

A “cell” or “host cell” refers to an individual cell or cell culture that can be or has been a recipient of any recombinant vector(s), isolated polynucleotide, or polypeptide. Host cells include progeny of a single host cell, and the progeny may not necessarily be completely identical (in morphology or in total DNA complement) to the original parent cell due to natural, accidental, or deliberate mutation and/or change. A host cell includes cells transfected or infected in vivo or in vitro with a recombinant vector or a polynucleotide of the invention. A host cell which comprises a recombinant vector of the invention is a recombinant host cell.

The term “about” or “approximately” means within an acceptable range for the particular value as determined by one of ordinary skill in the art, which will depend in part on how the value is measured or determined, e.g., the limitations of the measurement system. For example, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5 fold, and more preferably within 2 fold, of a value. Unless otherwise stated, the term ‘about’ means within an acceptable error range for the particular value, such as ±1-20%, preferably ±1-10% and more preferably ±1-5%.

Where a range of values is provided, it is understood that each intervening value, between the upper and lower limit of that range and any other stated or intervening value in that stated range is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either both of those included limits are also included in the invention.

Method of the invention are conveniently practiced by providing the compounds and/or compositions used in such method in the form of a kit. Such kit preferably contains the composition. Such a kit preferably contains instructions for the use thereof.

To gain a better understanding of the invention described herein, the following examples are set forth. It should be understood that these examples are for illustrative purposes only. Therefore, they should not limit the scope of this invention in anyway.

### EXAMPLES

### Metabolic Engineering

Metabolic engineering is a technique that targets specific pathways in the organism to adapt, enhance, or add a novel metabolite. This can be used as an alternate to the wild type biosynthesis of a higher order, and slower growth, organism such as a plant or animal, and produce the identical natural metabolite in a different host organism. The metabolites can be flavors, agricultural products, cosmetics, pharmaceuticals, and other high value small molecules or proteins. (Bhantna 2015). Early products such as Beta-Galactoside alpha-2,6 sialyltransferase as a glycosylation enzyme in Chinese Hamster Ovary cells for the production of erythropoietin, or D-Amino Acid Oxidase in A. Chrysogenum have been commercialized since the early 1990's. This technique shows promise for production of many high value molecules. The low cost, fast doubling times, and relative genetic simplicity make microorganisms the ideal target for metabolic engineering.

The anabolic metabolites are generated by first identifying the intrinsic processes that are present in the microorganism. Once a suitable common precursor is found, the metabolic pathway for the desired metabolite is then introduced into the organism. While the process of introducing the genes into the organism is important to the overall production of the metabolite, the actual techniques used will vary and should all result in the same product. The genes for the enzymes that will generate these metabolites will be inserted and expressed either constitutively or via an induction methodology.

Induction methods will grow the organism in its natural state, and then switch over to an alternate carbon source, that will refocus the resources of the microbe to generate the desired product. At this point the culture has grown close to its desired concentration, and then can move from a growth state, to a metabolic production state. Other than induction, the desired metabolite production can be optimized by diverting resources to the precursors for the introduced pathway from other less desirable products. This technique needs to be sure that vital systems remain intact.

Other foci of metabolic engineering include decreasing the cells catabolism, increasing the flux of carbon though the intended target pathway, optimizing, replacing, or adding better enzymes in the rate limiting steps of the process, decreasing the competitive pathways, or increasing the biomass of the production strain.

Problems with Cannabinoid Biosynthesis in Alternative Microorganisms:

Cannabinoid biosynthesis in microorganisms commonly utilized in broader biotechnology applications fail to sufficiently meet the needs required to mass produce purified cannabinoid products at scale. Escherichia coli (E. Coli) is unable to perform the post translational modifications required for the glycosylation steps of cannabinoid biosynthesis. Evidence suggests that Saccharomyces cerevisiae (S. Cerevisiae) as an organism can overcome the limitations presented with E. coli in an attempt to biosynthesize cannabinoids, but that yields from S. cerevisiae fail to reach levels practical for commercialization. Biosynthesis of small molecule isolates assayed by Lou and others have only shown cannabinoid yields in mg quantities with little evidence to suggest significantly higher yields are attainable (Lou et al., 2019).

Problems with Cell Free Cannabinoid Biosynthesis:

Existing literature demonstrates that CBD/THC cannabinoid production yields of 1 g/L have been synthesized with biotechnologies that perfuse sugar, oxygen and nutrients via stainless steel fermentation tanks (Valliere et al., 2019). Despite early evidence suggesting reasonable cannabinoid yields could be attainable through cell free biosynthesis platforms, challenges still remain in that individual proteins would still be required to be synthesized and extracted for each enzyme required in the process. Work performed by Valliere et al. Incorporates numerous individual enzymes that would each require biosynthesis and extraction. Therefore, the process of cell free biosynthesis proposed by Valliere and others does not solve the immediate need for a more cost effective method of cannabinoid biosynthesis.

### Description of the Technology

Cannabinoid production in P. pastoris overcomes the disadvantages of previous systems of cannabinoid production for commercial use falling into one or more of the following categories 1) extensive requirements for environmental resources, 2) limited production of cannabinoids in plant systems, 3) requirements for post-translational modifications not available in E. coli expression systems, 4) low solubility of intermediates in cannabinoid synthesis pathway, 5) low expression of starting materials in P. pastoris, 6) production of non-native cannabinoid and related molecules not found naturally in cannabis, and/or 7) lower biomass yield in other biosynthetic strains.

Pichia pastoris is synonymous with Komagataella phaffii, Komagataella pastoris, and Komagataella pseudopastoris for many uses and in the case of this patent.

In one example, described herein is a robust, scalable platform for the production of GPP. In one example, described herein is a robust, scalable platform for the production of CBGA.

### Example 1

In one example, described herein is a robust, scalable platform for the production GPP.

### Creation of a Stable Methylotrophic Yeast Strain Containing a Modified Erg20 Gene

Two stable transformations of the methylotrophic yeast P. pastoris were created through genetic recombination to generate either an insertion of the modified Erg20 gene or a replacement of the endogenous Erg20 gene.

In one embodiment of this technology a methylotrophic yeast strain (Bg10, Biogrammatics, Carlsbad, Calif.) was transformed with an Erg20 variant gene (SEQ ID NO:1) in an integrating vector with homology arms adjacent to the sites of the mutations which direct the mutation to the endogenous Erg20 location resulting in replacement. 5 μg of Erg20 replacement gene in a vector containing a gene for hygromycin B resistance was linearized via restriction enzyme digestion, transformed into cells lacking hygromycin B resistance through electroporation of the cell membrane, and grown on YPD agar containing hygromycin B at inhibitory concentrations to facilitate selection. Methylotrophic yeast cells that lacked successful incorporation of the integration plasmid, which included the hygromycin B resistance gene, died. Several colonies, with successful vector insertion, were picked and grown in BMGY media (10 g/L yeast extract, 20 g/L peptone, 0.1M potassium phosphate buffer pH 6.0, 1.34% w/v yeast nitrogen base, 4×10-5% biotin, 1% glycerol) at 28-30° C. All colonies were screened for successful integration by PCR. Clones that were validated via PCR were stored at −80° C. in glycerol stocks.

In another embodiment of this technology a methylotrophic yeast strain (Bg10, Biogrammatics, Carlsbad, Calif.) was transformed with an Erg20 variant gene in an integrating vector with a complete endogenous promoter and homology arms that allow for the incorporation of an additional copy of the Erg20 variant gene alongside the endogenous copy (SEQ ID NO:2).

5 μg of Erg20 duplication gene in a vector containing a gene for hygromycin B resistance was linearized via restriction enzyme digestion, transformed into cells lacking hygromycin B resistance through electroporation of the cell membrane, and grown on YPD agar containing hygromycin B at inhibitory concentrations to facilitate selection. Methylotrophic yeast cells that lacked successful incorporation of the integration plasmid, which included the hygromycin B resistance gene, died. Several colonies, with successful vector insertion, were picked and grown in BMGY media (10 g/L yeast extract, 20 g/L peptone, 0.1M potassium phosphate buffer pH 6.0, 1.34% w/v yeast nitrogen base, 4×10-5% biotin, 1% glycerol) at 28-30° C. All colonies were screened for successful integration by PCR. Clones that were validated via PCR were stored at −80° C. in glycerol stocks.

### Production of Erg20 in Methylotrophic Yeast

To initiate the Erg20 variant expression a fresh colony was generated from glycerol stock of Erg20 replacement or Erg20 duplication through streaking on an agar plate to generate single colonies. A single colony was then picked and grown in 5 mL of BMGY media overnight at 30° C. The overnight culture was then pelleted by centrifugation, washed with 10 mL phosphate buffered saline and re-pelleted. The cell pellet was then resuspended in 50 mL of BMGY and grown to an optical density (OD) of approximately 15.

Production of Erg20 variant production of GPP and FPP was analyzed. Cells were spun down, washed with PBS, and then the cell pellet was stored at −20° C. To each cell pellet, 0.5 mL of 2-propanol: 100 mM NH₄HCO₃, pH 7.4 (1:1 v/v), was added, cells were then sonicated, and 300 mL of the resulting cell homogenate was used for further preparation. Subsequently, 1.0 mL of methanol was added for deproteinization, and then mixture was cooled (−20° C.) for 10 min. The samples were then centrifuged for 10 min at 14,000×g at 4° C. After centrifugation, supernatants were transferred to glass tubes and dried under a stream of nitrogen at 40° C. The residues were then dissolved in 100 μL acetonitrile:water (1:1, v/v) and 5 μL of this solution was injected into the

LC-MS/MS system to confirm and quantitate the production of GPP and the ratio of GPP:FPP.

### Example 2

In one example, described herein is a robust, scalable platform for the production GPP.

### Biosynthetic Production of Cannabigerolic Acid (CBGA) and Related Cannabinoids in Methylotrophic Yeast

This technology embodies a system of converting glucose or other readily available sugars such as, but not limited to dextrose, to cannabinoids in the methylotrophic yeast Pichia pastoris. Cannabinoids act on the endogenous endocannabinoid system in humans resulting in changes in psychological behavior through modulation of neurotransmitters and brain chemistry. Members of this family are synthesized naturally in humans as well as in plants and have been shown to have a variety of medicinal and therapeutic value making them key targets of the pharmaceutical industry.

Current cannabinoid production is limited to organic synthesis, limited production in some microorganisms, and extraction from natural sources. Current methods of cannabinoid production have significant drawbacks in their requirement for expensive chemical precursors, biologically incompatible solvents, limited yields of desired products, stereochemical racemization of products, reduced potency compared to naturally produced cannabinoids, and limited yields. Isolation of cannabinoids from natural sources also suffer due to significant requirements for arable land, large amounts of water, long growth times, mixed cannabinoid production, and low yields as an overall percentage of plant mass. Another limitation to the plant production of cannabinoids is the prevalence of similar compounds.

Disclosed herein are strategies for the production of chemically pure cannabinoid molecules using the methylotrophic yeast P. pastoris (Komagataella phaffi). Through genetic engineering of this microorganism along with directed evolution and generation of novel proteins with specific enzymatic function we can achieve the production of the desired cannabinoid compound from a variety of sugars (glucose, galactose, fructose, sucrose, and naturally occurring mixtures containing high sugar contents).

Through genetic engineering the inventors have reconstituted a pathway for the production of cannabigerolic (CBGA) acid into the methylotrophic yeast P. pastoris. Production of CBGA is the crucial intermediate in the synthesis of pharmaceutically active cannabinoid molecules (including but not limited to THCA, CBDA, CBCA, etc.). While synthesized naturally in the acid containing forms, most cannabinoid molecules are only active in the decarboxylated forms which are achieved through heating of the molecules following their isolation.

The biosynthetic route for the production of CBGA in P. pastoris requires the production of hexanoyl-CoA from acetyl-CoA precursor molecules through a series of enzymatic activities. In the natural plant this process is achieved through the conversion of an available pool of hexanoic acid through the use of an acyl-activating enzyme (AAE). Most yeasts, including methyolotrophic yeasts like P. pastoris, lack an endogenous pool of short and medium chain fatty acids requiring the introduction of a heterologous system for the production of the required hexanoyl-CoA precursor molecule (our system addresses this issue below). Hexanoyl-CoA is then combined with three molecules of malonoyl-CoAto form olivetolic acid (OA) through a prenylation event catalyzed by a preny transferase enzyme followed by a cyclization event catalyzed by an olevitolic acid cyclase. OA is then combined with a molecule of geranyl pyrophosphate (GPP) through the activity of an aromatic prenyl transferase to produce CBGA. Subsequent transformation of CBGA into pharmaceutically relevant cannabinoids is accomplished by one of a number of synthase proteins specific to the final cannabinoid product.

Embodiments of this technology include a heterologous pathway for the production of CBGA. The inventors, through genetic engineering, have introduced the pathway required for the conversion of hexanoyl-CoA to olevitolic acid. This pathway begins by a heterologous polyketide synthase (CsTKS) combining one molecule of hexanoyl-CoA with three molecules of malonoyl-CoA to generate 3,5,7-trioxododecanoyl-CoA. 3,5,7-trioxododecanoyl-CoA is cyclized through the activity of an olevitolic acid cyclase (CsOAC) enzyme releasing olevitolic acid and a molecule of coenzyme A. An aromatic prenyltransferase (CsPT4) catalyzes the final step in the reaction pathway combining the olevitolic acid produced in the previous steps with GPP resulting in the production of CBGA.

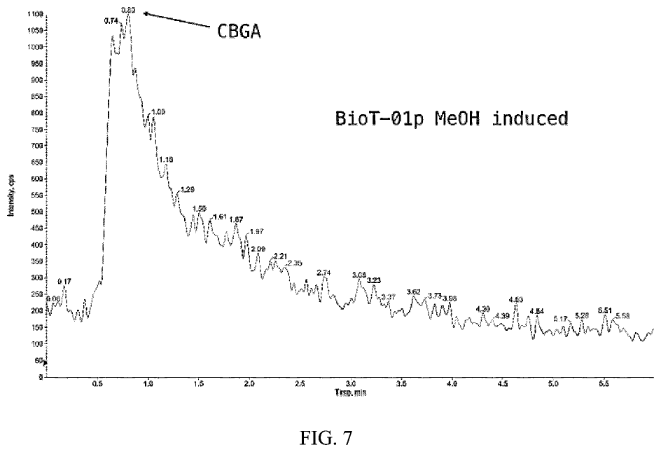

FIG. 7 depicts Analytical results for CBGA production. LC/MS of GBGA generated from a P. pastoris strain that had been modified to contain the biosynthetic cannabinoid pathway. The sample was separated on a C8 reverse phase column using water and acetonitrile as aqueous and organic phases in a gradient. The detection method was a total ion current, and the CBGA peak appears at the 0.80 min mark.

FIG. 5 depicts Quantification of MeOH induced P. pastoris CBGA levels. Calibration curve and analysis of LC/MS data showing the quantification of the CBGA produced in P. pastoris. Panel A depicts CBGA injected into MeOH grown yeast. Panel B should a graph of Peak Area versus MeOH CBGA. Panel C shows a summary of data produced in P. pastoris.

In one example, the AOX1 promoter, which responds to methanol induction, may be used.

Materials

Culture Medium:

Prior to all transformation procedures, P. pastoris strains are cultured in YPD medium (1% yeast extract, 2% peptone, and 2% dextrose). Growth of P. pastoris in liquid media and on agar media plates is at 30° C. unless otherwise specified.

BMGY Medium:

1% yeast extract

2% peptone

100 mM potassium phosphate, pH 6.0

1.34% yeast nitrogen base (YNB)

4×10-5% biotin

1% glycerol

BMMY Medium:

1% yeast extract

2% peptone

100 mM potassium phosphate, pH 6.0

1.34% yeast nitrogen base (YNB)

4×10-5% biotin

0.5-1% methanol

Electroporation:

1. H₂O (1 L).

2. 1 M sorbitol (100 mL).

3. YND agar plates: 0.67% YNB, 2% glucose, and 2% agar (0.5 L/˜25 plates).

4. 1M DTT (2.5 mL).

5. YPD medium (100 mL) with 20 mL 1 M HEPES buffer (pH 8.0).

6. Electroporation instrument (e.g., BTX Electro Cell Manipulator 600, BTX, San Diego, Calif.; Bio-Rad Gene Pulser, Bio-Rad, Hercules, Calif.; Electroporator II, Invitrogen, San Diego, Calif.).

7. Sterile electroporation cuvettes.

All solutions should be autoclaved except the DTT and HEPES solutions, which should be filter sterilized.

Rapid Heat-Shock/Electroporation:

1. BEDS solution (9 mL): 10 mM bicine-NaOH (pH 8.3), 3% ethylene glycol, and 5% dimethyl sulfoxide (DMSO).

2. 1 M sorbitol supplemented with 1 mL 1.0M DTT.

### Methods

This procedure is a modified version of that described by Becker and Guarente. Electroporation was conducted at 1150V, 600 ohms, and 25 uF with 4-5 ms pulses.

### Preparation of Competent Cells:

1. Inoculate a 10 mL YPD culture with a single fresh P. pastoris colony of the strain to be transformed from an agar plate and grow overnight with shaking.

2. In the morning, use the overnight culture to inoculate a 500 mL YPD culture in a 2.8-L Fernback culture flask to a starting OD₆₀₀ of 0.1 and grow to an OD₆₀₀ of 1.0 (see Note 1).

3. Harvest the culture by centrifugation at 2000 g at 4° C., and suspend the cells in 100 mL of YPD medium plus HEPES.

4. Add 2.5 mL of 1 M DTT and gently mix.

5. Incubate at 30° C. for 15 min.

6. Bring to 500 mL with cold water. Wash by centrifugation at 4° C. once in 250 mL of cold water, once in 20 mL of cold 1 M sorbitol and resuspend in 0.5 mL of cold 1 M sorbitol. (Final volume including cells will be 1.0 to 1.5 mL.)

7. For highest frequencies, transform the cells directly without freezing as described below.

8. To freeze competent cells, distribute in 40 μL aliquots to sterile 1.5 mL microcentrifuge tubes, and place the tubes in a −80° C. freezer until use.

### Rapid Heat-Shock/Electroporation:

1. Grow 5 mL culture of P. pastoris cells in YPD overnight with shaking.

2. The next morning, dilute the overnight culture to an OD₆₀₀ of 0.15 to 0.20 in a volume of 50 mL YPD medium in a flask large enough to provide good aeration.

3. Grow to an OD₆₀₀ of 0.8 to 1.0 with shaking (4-5 h).

4. Centrifuge cells at 500 g for 5 min at room temperature then decant supernatant.

5. Suspend cells in 9 mL of ice-cold BEDS solution supplemented with DTT.

6. Incubate the cell suspension for 5 min at with shaking at 30° C.

7. Centrifuge cells at 500 g for 5 min at room temperature and resuspend in 1 mL of BEDS (without DTT).

8. Immediately perform electroporation as described in below, or freeze cells in small aliquots at −80° C.

### Electroporation:

1. Mix up to 10 μg of DNA sample in no more than 5 μL total volume of water or TE buffer to a tube containing 40 μL of frozen or fresh competent cells and transfer to a 2-mm gap electroporation cuvette held on ice (see Notes 2 and 3).

2. Pulse cells according to the parameters suggested for yeast by the manufacturer of the specific electroporation instrument being used (1150V, 600 ohms, and 25 uF with 4-5 ms pulses, see Note 4).

3. Immediately add 1 mL of cold 1M sorbitol and transfer the cuvette contents to a sterile 1.5-mL microcentrifuge tube.

4. Spread selected aliquots onto agar plates containing selective medium and incubate for 2 to 4 days (see Note 5).

UPP Promoter Growth:

1. Select a single colony from agar plate (if available) or streak a previously stored glycerol stock onto an agar plate containing the appropriate selection media to generate a single colony to select.

2. Add selected colony to 5 mL of BMGY growth media and incubate while shaking overnight.

3. Spin down overnight culture at 2,000 g for 5 minutes.

4. Remove spent BMGY media and resuspend cells in phosphate buffered saline (PBS).

5. Spin down culture, remove PBS, resuspend cells in 50 mL of fresh BMGY media.

6. Grow culture until OD₆₀₀ of approximately 15 has been reached.

7. Spin down culture at 2,000 g for 5 minutes.

8. Remove spent BMGY media and store cells at −80° C. for LC-MS/MS analysis.

### AOX1 Promoter Growth:

1. Select a single colony from agar plate (if available) or streak a previously stored glycerol stock onto an agar plate containing the appropriate selection media to generate a single colony to select.

2. Add selected colony to 5 mL of BMGY growth media and incubate while shaking overnight.

3. Spin down overnight culture at 2,000 g for 5 minutes.

4. Remove spent BMGY media and resuspend cells in phosphate buffered saline (PBS).

5. Spin down culture, remove PBS, resuspend cells in 50 mL of fresh BMGY media.

6. Grow culture until OD₆₀₀ of approximately 15 has been reached.

7. Spin down culture at 2,000 g for 5 minutes.

8. Remove spent BMGY media and resuspend cells in phosphate buffered saline (PBS).

9. Spin down culture, remove PBS, resuspend cells in 50 mL of fresh BMMY media.

10. After 24 hrs incubation at 30° C., add additional methanol to a final concentration of 0.5-1%.

11. Supplement methanol at 12 hr time points until a total incubation time of 60-72 hrs has been reached.

12. Spin down culture at 2,000 g for 5 minutes.

13. Remove spent BMMY media and store cells at −80° C. for LC-MS/MS analysis.

### LC-MS/MS Analysis GPP/FPP:

1. Add 0.5 mL of 1:1 (v/v) 2-propanol: 100 mM NH₄HCO₃, pH 7.4.

2. Sonicate cell mixture to disrupt cell membranes.

3. Add 10 mL methanol and cool mixture to −20° C. for 10 minutes.

4. Centrifuge samples for 10 minutes at 14,000 g at 4° C.

5. Transfer supernatant to glass tubes.

6. Dry under a stream of nitrogen at 40° C.

7. Dissolve residue in 100 μL 1:1 (v/v) acetonitrile:water.

8. Inject 5 μL of the solution into Zorbax 50×2.1 mm column at 20° C. on an Agilent HPLC 1100, API 4000 Qtrap Mass Spectrometer.

### LC-MS/MS Analysis CBGA:

1. Lyse cells using any mechanical technique.

2. Add 1 mL of 4M KCl, pH2.0 to each 1 mL of cell lysate.

3. Add 1-2 mL of ethyl acetate for each 1 mL of cell lysate.

4. Rigorously mix for 1 min.

5. Centrifuge the mixture for 5 minutes at 1000 g at 4° C.

6. Remove the top ethyl acetate layer and transfer to glass tubes.

7. Dry under a stream of nitrogen at 40° C.

8. Dissolve residue in 100 μL 1:1 (v/v) acetonitrile:water.

9. Inject 5 μL of the solution into Zorbax 50×2.1 mm column at 20° C. on an Agilent HPLC 1100, API 4000 Qtrap Mass Spectrometer.

### Notes:

1. One OD₆₀₀ unit of P. pastoris culture equals approx. 5×10⁷ cells.

2. If using Invitrogen electroporation cuvettes, the volume of cells should be 80 μL. If using BRL 1.5-mm gap chambers, the volume of cells should be 20 μL.

3. Cell competence decreases very rapidly after cells thaw even if the cells are held on ice. Therefore, adding DNA to frozen samples is critical. To transform large numbers of samples, it is convenient to process them in groups of about six at a time.

4. In general, procedures described for S. cerevisiae also work well for P. pastoris. Thus, if your instrument is not listed, use the protocol recommended for electroporation of S. cerevisiae with your instrument. See technical literature provided by manufacturers for information on the use of specific electroporation devices.

5. P. pastoris cells are flocculent (i.e., tend to grow in multi-cell clumps). As a result, transformant colonies are frequently composed of more than one transformed strain. To avoid the problem of mixed colonies, pick and re-streak them for single colonies on selective medium at least once before proceeding with further analysis. When looking for gene replacement transformants by replica plate screening for a recessive phenotype (e.g., AOX1 gene replacements with a recessive methanol-utilization slow or Muts phenotype), colonies should be recovered from the transformation plates, suspended in water, and re-plated on selective medium before screening.

### Transformation and Plasmid Expansion

Equipment:

Shaking incubator at 37° C.

Stationary incubator at 37° C.

Water bath at 42° C.

Ice bucket filled with ice

Microcentrifuge tubes

Sterile spreading device

Materials:

LB agar plate (10 g/L peptone, 5 g/L yeast extract, 5 g/l NaCl, 12 g/L agar)

SOC media (20 g/L tryptone, 5 g/L yeast extract, 0.5 g/L NaCl).

Competent E. coli cells (DH5-alpha)

Ampicillin

DNA plasmid for transformation

### Method:

1. Take competent cells out of −80° C. and thaw on ice (approximately 20-30 mins).

2. Remove agar plates (containing the appropriate antibiotic) from storage at 4° C. and let warm up to room temperature and then (optional) incubate in 37° C. incubator.

3. Mix 1-5 μL of DNA (usually 10 pg-100 ng) into 20-50 μL of competent cells in a microcentrifuge or falcon tube. GENTLY mix by flicking the bottom of the tube with your finger a few times.

4. Incubate the competent cell/DNA mixture on ice for 20-30 mins.

5. Heat shock each transformation tube by placing the bottom ½ to ⅔ of the tube into a 42° C. water bath for 30-60 secs (45 seconds is usually ideal, but this varies depending on the competent cells you are using).

6. Put the tubes back on ice for 2 min.

7. Add 250-1,000 μl SOC media (without antibiotic) to the bacteria and grow in 37° C. shaking incubator for 45 min.

8. Plate some 50 μL of the transformation onto a 10 cm LB agar plate containing the appropriate antibiotic.

9. Incubate plates at 37° C. overnight.

The embodiments described herein are intended to be examples only. Alterations, modifications and variations can be effected to the particular embodiments by those of skill in the art. The scope of the claims should not be limited by the particular embodiments set forth herein, but should be construed in a manner consistent with the specification as a whole.

All publications, patents and patent applications mentioned in this Specification are indicative of the level of skill those skilled in the art to which this invention pertains and are herein incorporated by reference to the same extent as if each individual publication patent, or patent application was specifically and individually indicated to be incorporated by reference.

The invention being thus described, it will be obvious that the same may be varied in many ways. Such variations are not to be regarded as a departure from the spirit and scope of the invention, and all such modification as would be obvious to one skilled in the art are intended to be included within the scope of the following claims.

What is claimed is:

1. A method of producing geranyl pyrophosphate (GPP) in a methylotrophic yeast host cell, comprising:
(i) introducing a first polynucleotide encoding a modified Erg20 polypeptide into the methyltrophic yeast cell, wherein the modified Erg20 polypeptide comprises a F98W and N128W amino acid substitution (Erg20 (F98W/N128W)); and,
(ii) culturing the methylotrophic yeast host cell under conditions sufficient for GPP production.

2. The method of claim 1, wherein the methylotrophic yeast host cell is Pichia pastoris (Komagataella phaffii).

3. The method of claim 1, wherein the first polynucleotide encoding the Erg20 (F98W/N128W) polypeptide comprises: a) a nucleotide sequence of SEQ ID NO:1 or SEQ ID: 2; b) a nucleic acid having at least 70% identity to the nucleic acid of a), c) a nucleic acid that hybridizes, optionally under conditions of high stringency, with the complementary strand of the nucleic acid of a), d) a nucleic acid that differs from a) by one or more nucleotides that are substituted, deleted, and/or inserted; or e) a derivative or variant of a), b), c), or d).

4. The method of claim 1, wherein the first polynucleotide encoding an Erg20 (F98W/N128W) polypeptide consists of: a) a nucleotide sequence of SEQ ID NO: 1 or SEQ ID NO:2; b) a nucleic acid having at least 70% identity to the nucleic acid of a), c) a nucleic acid that hybridizes, optionally under conditions of high stringency, with the complementary strand of the nucleic acid of a), d) a nucleic acid that differs from a) by one or more nucleotides that are substituted, deleted, and/or inserted; or e) a derivative or variant of a), b), c), or d).

5. An isolated polynucleotide comprising: a) a nucleotide sequence of SEQ ID NO:1 or SEQ ID NO:2, b) a nucleotide sequence having at least 70% identity to the nucleotide sequence of a), c) a nucleotide sequence that hybridizes with the complementary strand of the nucleic acid of a), d) a nucleotide sequence that differs from a) by one or more nucleotides that are substituted, deleted, and/or inserted; or e) a derivative of a), b), c), or d).

6. The isolated polynucleotide of claim 5, wherein the isolated polynucleotide consists of: a) a nucleotide sequence set forth in SEQ ID NO:1 or SEQ ID NO:2, b) a nucleotide sequence having at least 70% identity to the nucleotide sequence of a), c) a nucleotide sequence that hybridizes with the complementary strand of the nucleic acid of a), d) a nucleotide sequence that differs from a) by one or more nucleotides that are substituted, deleted, and/or inserted; or e) a derivative of a), b), c), or d).

7. The isolated polynucleotide of claim 5, wherein the polynucleotide encodes an Erg20 (F98W/N128W) polypeptide.

8. The isolated polynucleotide of claim 6, wherein the polynucleotide encodes an Erg20 (F98W/N128W) polypeptide.

9. A method of producing cannabigerolic acid (CBGA) in a methylotrophic yeast host cell comprising:
(i) introducing into the methylotrophic yeast host cell:
(a) a first polynucleotide encoding an olevitiolic synthase polypeptide, a second polynucleotide encoding an olevitiolic acid cyclase polypeptide, and a third polynucleotide encoding an aromatic prenyl transferase;
(b) a fourth polynucleotide encoding i) an olevitiolic synthase polypeptide and ii) an olevitiolic acid cyclase polypeptide, and the third polynucleotide encoding an aromatic prenyl transferase; or
(c) the first polynucleotide encoding an olevitiolic synthase polypeptide, and a fifth polynucleotide encoding i) an olevitiolic acid cyclase polypeptide and ii) an acormatic prenyl transferase; and
(ii) culturing the methylotrophic yeast host cell under conditions sufficient for CBGA production.

10. The method of claim 9, wherein the methylotrophic yeast host cell is from Pichia pastoris (Komagataella phaffii).

11. The method of claim 9, wherein the conditions sufficient for CBGA production comprise methanol induction.

12. The method of claim 9, wherein the first polynucleotide encoding olevitiolic synthase polypeptide comprises: a) a nucleotide sequence of SEQ ID NO:6; b) a nucleic acid having at least 70% identity to the nucleic acid of a), c) a nucleic acid that hybridizes with the complementary strand of the nucleic acid of a), d) a nucleic acid that differs from a) by one or more nucleotides that are substituted, deleted, and/or inserted; or e) a derivative of a), b), c), or d).

13. The method of claim 12, wherein the first polynucleotide encoding olevitiolic synthase polypeptide consists of: a) a nucleotide sequence of SEQ ID NO:6; b) a nucleic acid having at least 70% identity to the nucleic acid of a), c) a nucleic acid that hybridizes with the complementary strand of the nucleic acid of a), d) a nucleic acid that differs from a) by one or more nucleotides that are substituted, deleted, and/or inserted; or e) a derivative of a), b), c), or d).

14. The method of claim 9, wherein the second polynucleotide encoding olevitiolic acid cyclase polypeptide comprises: a) a nucleotide sequence of SEQ ID NO:9; b) a nucleic acid having at least 70% identity to the nucleic acid of a), c) a nucleic acid that hybridizes with the complementary strand of the nucleic acid of a), d) a nucleic acid that differs from a) by one or more nucleotides that are substituted, deleted, and/or inserted; or e) a derivative of a), b), c), or d).

15. The method of claim 14, wherein the second polynucleotide encoding olevitiolic acid cyclase polypeptide consists of: a) a nucleotide sequence of SEQ ID NO:9; b) a nucleic acid having at least 70% identity to the nucleic acid of a), c) a nucleic acid that hybridizes with the complementary strand of the nucleic acid of a), d) a nucleic acid that differs from a) by one or more nucleotides that are substituted, deleted, and/or inserted; or e) a derivative of a), b), c), or d).

16. The method of claim 9, wherein the third polynucleotide encoding the acormatic prenyl transferase comprises: a) a nucleotide sequence of SEQ ID NO:8; b) a nucleic acid having at least 70% identity to the nucleic acid of a), c) a nucleic acid that hybridizes with the complementary strand of the nucleic acid of a), d) a nucleic acid that differs from a) by one or more nucleotides that are substituted, deleted, and/or inserted; or e) a derivative of a), b), c), or d).

17. The method of claim 16, wherein the third polynucleotide encoding the acormatic prenyl transferase consists of: a) a nucleotide sequence of SEQ ID NO:8; b) a nucleic acid having at least 70% identity to the nucleic acid of a), c) a nucleic acid that hybridizes with the complementary strand of the nucleic acid of a), d) a nucleic acid that differs from a) by one or more nucleotides that are substituted, deleted, and/or inserted; or e) a derivative of a), b), c), or d).

18. The method of claim 8, wherein the fourth polynucleotide encoding olevitiolic synthase polypeptide and encoding olevitiolic acid cyclase polypeptide comprises: a) a nucleotide sequence of SEQ ID NO:3; b) a nucleic acid having at least 70% identity to the nucleic acid of a), c) a nucleic acid that hybridizes with the complementary strand of the nucleic acid of a), d) a nucleic acid that differs from a) by one or more nucleotides that are substituted, deleted, and/or inserted; or e) a derivative of a), b), c), or d).

19. The method of claim 18, wherein the fourth polynucleotide encoding olevitiolic synthase polypeptide and encoding olevitiolic acid cyclase polypeptide consists of: a) a nucleotide sequence of SEQ ID NO:3; b) a nucleic acid having at least 70% identity to the nucleic acid of a), c) a nucleic acid that hybridizes with the complementary strand of the nucleic acid of a), d) a nucleic acid that differs from a) by one or more nucleotides that are substituted, deleted, and/or inserted; or e) a derivative of a), b), c), or d).

20. The method of claim 8, wherein fifth polynucleotide encoding olevitiolic acid cyclase polypeptide and encoding aromatic prenyl transferase comprises: a) a nucleotide sequence of SEQ ID NO:10; b) a nucleic acid having at least 70% identity to the nucleic acid of a), c) a nucleic acid that hybridizes with the complementary strand of the nucleic acid of a), d) a nucleic acid that differs from a) by one or more nucleotides that are substituted, deleted, and/or inserted; or e) a derivative of a), b), c), or d).

21. The method of claim 8, wherein fifth polynucleotide encoding olevitiolic acid cyclase polypeptide and encoding aromatic prenyl transferase consists of: a) a nucleotide sequence of SEQ ID NO:10; b) a nucleic acid having at least 70% identity to the nucleic acid of a), c) a nucleic acid that hybridizes with the complementary strand of the nucleic acid of a), d) a nucleic acid that differs from a) by one or more nucleotides that are substituted, deleted, and/or inserted; or e) a derivative of a), b), c), or d).

22. The method of claim 12, wherein in step (c) the polynucleotide hybridizes with the complementary strand of the nucleic acid of a) under conditions of high stringency.

23. The method of claim 13, wherein in step (c) the polynucleotide hybridizes with the complementary strand of the nucleic acid of a) under conditions of high stringency.

24. An expression vector comprising an isolated polynucleotide, comprising: a) a nucleotide sequence of SEQ ID NO:1, SEQ ID NO:2, SEQ ID NO:3, SEQ ID NO:6, SEQ ID NO:8, SEQ ID NO:9, or SEQ ID NO:10, b) a nucleotide sequence having at least 70% identity to the nucleotide sequence of a), c) a nucleotide sequence that hybridizes, optionally under conditions of high stringency, with the complementary strand of the nucleic acid of a), d) a nucleotide sequence that differs from a) by one or more nucleotides that are substituted, deleted, and/or inserted; or e) a derivative of a), b), c), or d).

25. A methylotrophic yeast host cell comprising:
(i) an expression vector of claim 24;
(ii) a nucleotide sequence set forth in SEQ ID NO:1, SEQ ID NO:2, SEQ ID NO:3, SEQ ID NO:6, SEQ ID NO:8, SEQ ID NO:9, or SEQ ID NO:10;
(iii) a nucleotide sequence having at least 70% identity to the nucleotide sequence of (ii);
(iv) a nucleotide sequence that hybridizes with the complementary strand of the nucleic acid of (ii);
(v) a nucleotide sequence that differs from (ii) by one or more nucleotides that are substituted, deleted, and/or inserted; or
(vi) a derivative of (ii), (iii), (iv), or (v).

26. The methylotrophic yeast host cell of claim 25, wherein the methylotrophic yeast host cell is from Pichia pastoris (Komagataella phaffii).


SEQUENCE LISTING (10 sequences)

SEQ ID NO: 1 - DNA, 2001 bp, Pichia pastoris
agtctcaagactggagactcgctctttatagaaatagatatagccttttactattaattccattttagtcatgctgtgttatattttgtagacatgatataatcactgtatattttcaactttgatgtgcccttaaacgttaacacgctcaagctcctctctacctgccttggtgtacaggtattcattgtagtctctagcttgagcccacacgttccaaacgatgatacctggaataatcacatacaaagcctgagctttggttcttctccaaacgttgaaaatggctctttccgatgatcctttcaaaggagatccgacgtaaggggagatggtgtaggtagtgacgttcttttgcttgggggatccaatgaatccccaccagcccatgtaagcacctggatgtcctgccattcttaatgtagttttagggtaagtagatggtagtagaatttcccgtattatattcgaggccatctgattggtgataattccgttctgaaatattcgcagggaaacttaggttggtagcagtgatacctgtaactaccttgcagagataatcaccagctaataaagtatggccttggccctttcgcatagagaatggtcaagacgtacagaactatgagggcccaacactgtttaaactttagtttggtgccaacttatgatttaaaaagccagacaattttaattccccaaacagacctctaattttgcccgaataatgccaattaacattctagaaacttcgattaactatatatttcataaaccatgacctttttccgccaatatcctcatatttcagcctaaggaagactctcctccttttagcctacaacgaacttgccccataaatacaccaagcgcgaccctcgttcatcgtttaactcgtgttttcttctcacctttcgaactagtagaaatcaaatgtccaaagaagtagcagctaagaacaaagtgaagttccttgcgttctttccagacctcgtgtttgagctggaagtcatgctggaaaattacggcatgcctgctgatgcaatcaactggttcaagagatccttgaactacaacactcctggtggaaaactcaacagaggtatctctgttgtcgacacctatgtcattctcaaggggtacaagtcatgggacgagttaagtgccgaagagttcaagaaagctgctattttaggatggtgtgtcgaattgctacaagcctactggttggttgctgatgatatgatggaccaatctattaccagaagaggtcaaccatgttggtacaaggttgagaacgtcggtaacattgctatttgggactctttcatgttggagggtgccatctacaagattttgagaaagtacttcaagaaggagtcttactacgccgacttgttggacttgttgcacgaggttactttccaaactgaattgggtcaattgttggacttgatcactgctccagaagaccacgttgatctttccaagtttacccctgctaaacactctttcattgtcatcttcaagactgcctactattcattctaccttcctgttgttatggccatgtacttgtctggtattacccatgaaaaggatttgcagcaggctgagcacatcttgattccattgggtgagtacttccaaattcaagatgactacttggactgtttcggtaagccagaagatattggtaagattggaactgacatccaggacaataagtgttcttgggtgattaaccaagctctgagattggcctctccagagcaaagacaaattcttgatgagaactacggaagaaaagatgccgacaaggaggcaaagtgcaaggaagtttttgaccaacttgacattgctggtaagtacaaggcttacgaggaaaacatcggtaaggagttgcaaaagagaattgctgacactgaggaggatcgtggattcaagaaggaagttttccaggtctttttcgacaagatctacaagagaaccaaatag

SEQ ID NO: 2 - DNA, 1583 bp, Pichia pastoris
aagatgtttttgtttagttgactataagttaattttttatattaatttgcactctgtccttcactaaactttagtttggtgccaacttatgatttaaaaagccagacaattttaattccccaaacagacctctaattttgcccgaataatgccaattaacattctagaaacttcgattaactatatatttcataaaccatgacctttttccgccaatatcctcatatttcagcctaaggaagactctcctccttttagcctacaacgaacttgccccataaatacaccaagcgcgaccctcgttcatcgtttaactcgtgttagagacctattccagacttcagatctccaaaaacaggactatatcataatttggcacgtttgaatcttccccatcctgaagcggtatttgagatcaactacttcagagaaaaccctcatgctttttacacattggctgacgaactttaccctgggagggtctcattcttctcacctttcgaactagtagaaatcaaatgtccaaagaagtagcagctaagaacaaagtgaagttccttgcgttctttccagacctcgtgtttgagctggaagtcatgctggaaaattacggcatgcctgctgatgcaatcaactggttcaagagatccttgaactacaacactcctggtggaaaactcaacagaggtatctctgttgtcgacacctatgtcattctcaaggggtacaagtcatgggacgagttaagtgccgaagagttcaagaaagctgctattttaggatggtgtgtcgaattgctacaagcctactggttggttgctgatgatatgatggaccaatctattaccagaagaggtcaaccatgttggtacaaggttgagaacgtcggtaacattgctatttgggactctttcatgttggagggtgccatctacaagattttgagaaagtacttcaagaaggagtcttactacgccgacttgttggacttgttgcacgaggttactttccaaactgaattgggtcaattgttggacttgatcactgctccagaagaccacgttgatctttccaagtttacccctgctaaacactctttcattgtcatcttcaagactgcctactattcattctaccttcctgttgttatggccatgtacttgtctggtattacccatgaaaaggatttgcagcaggctgagcacatcttgattccattgggtgagtacttccaaattcaagatgactacttggactgtttcggtaagccagaagatattggtaagattggaactgacatccaggacaataagtgttcttgggtgattaaccaagctctgagattggcctctccagagcaaagacaaattcttgatgagaactacggaagaaaagatgccgacaaggaggcaaagtgcaaggaagtttttgaccaacttgacattgctggtaagtacaaggcttacgaggaaaacatcggtaaggagttgcaaaagagaattgctgacactgaggaggatcgtggattcaagaaggaagttttccaggtctttttcgacaagatctacaagagaaccaaatag

SEQ ID NO: 3 - DNA, 3987 bp, Cannabis sativa
gatctaacatccaaagacgaaaggttgaatgaaacctttttgccatccgacatccacaggtccattctcacacataagtgccaaacgcaacaggaggggatacactagcagcagaccgttgcaaacgcaggacctccactcctcttctcctcaacacccacttttgccatcgaaaaaccagcccagttattgggcttgattggagctcgctcattccaattccttctattaggctactaacaccatgactttattagcctgtctatcctggcccccctggcgaggttcatgtttgtttatttccgaatgcaacaagctccgcattacacccgaacatcactccagatgagggctttctgagtgtggggtcaaatagtttcatgttccccaaatggcccaaaactgacagtttaaacgctgtcttggaacctaatatgacaaaagcgtgatctcatccaagatgaactaagtttggttcgttgaaatgctaacggccagttggtcaaaaagaaacttccaaaagtcggcataccgtttgtcttgtttggtattgattgacgaatgctcaaaaataatctcattaatgcttagcgcagtctctctatcgcttctgaaccccggtgcacctgtgccgaaacgcaaatggggaaacacccgctttttggatgattatgcattgtctccacattgtatgcttccaagattctggtgggaatactgctgatagcctaacgttcatgatcaaaatttaactgttctaacccctacttgacagcaatatataaacagaaggaagctgccctgtcttaaacctttttttttatcatcattattagcttactttcataattgcgactggttccaattgacaagcttttgattttaacgacttttaacgacaacttgagaagatcaaaaaacaactaattattcgaaacgatgcatcatcatcatcaccattcctccggtagagagaacttgtacttccaaggtatgtctgctggttctgaccaaatcgaaggttccccacaccacgagtctgataactccattgctaccaagattttgaactttggccatacgtgttggaaattgcaaagaccttacgttgtcaagggtatgatatcgattgcttgtggtttgtttggtagagagttgttcaacaacagacatttgttctcttggggtttgatgtggaaggctttttttgcccttgttcctattctttctttcaacttttttgccgctatcatgaaccagatttacgatgttgatattgacagaatcaacaagccagaccttccattggtttctggtgagatgtccattgagactgcttggattctgtcaattatagttgctttgactggtttgattgtcactatcaagctgaaatctgctccactgtttgttttcatttacatctttggtatttttgctggttttgcctattcggttcctccaattagatggaagcaatatccattcaccaactttctgatcacaatttcctctcacgttggtttggctttcacaagctactccgctactacctctgctttgggtcttccattcgtgtggcgtccagccttttctttcatcattgctttcatgactgtcatgggtatgaccattgcttttgccaaggacatttctgatatcgaaggtgacgctaagtacggtgtctcgaccgttgctaccaagttgggtgccagaaatatgactttcgttgtttctggtgttttgctgttgaactacctggtgagtatttcaattggtattatctggccccaagtcttcaagtccaacattatgattctatctcacgctattttggctttctgtttgatcttccaaaccagagagctggctttggccaactacgcttccgctccatccagacaattctttgaattcatttggctattgtactacgccgagtacttcgtttatgttttcatctaatgaaatgtatttaatttgatattaagtaaatgaatgattatgactttatgaattcgcaatgttttctccttgattatttctgtattgtattggaatgattatagaatactcatatattgattatagtattagcacataaaacgtttgttgttaaactcacttccgtacgcaaccatttctatttctagctatcttgataaggtgatctaacatccaaagacgaaaggttgaatgaaacctttttgccatccgacatccacaggtccattctcacacataagtgccaaacgcaacaggaggggatacactagcagcagaccgttgcaaacgcaggacctccactcctcttctcctcaacacccacttttgccatcgaaaaaccagcccagttattgggcttgattggagctcgctcattccaattccttctattaggctactaacaccatgactttattagcctgtctatcctggcccccctggcgaggttcatgtttgtttatttccgaatgcaacaagctccgcattacacccgaacatcactccagatgagggctttctgagtgtggggtcaaatagtttcatgttccccaaatggcccaaaactgacagtttagagacctattccagacttcagatctccaaaaacaggactatatcataatttggcacgtttgaatcttccccatcctgaagcggtatttgagatcaactacttcagagaaaaccctcatgctttttacacattggctgacgaactttaccctgggagggtctcaaaacgctgtcttggaacctaatatgacaaaagcgtgatctcatccaagatgaactaagtttggttcgttgaaatgctaacggccagttggtcaaaaagaaacttccaaaagtcggcataccgtttgtcttgtttggtattgattgacgaatgctcaaaaataatctcattaatgcttagcgcagtctctctatcgcttctgaaccccggtgcacctgtgccgaaacgcaaatggggaaacacccgctttttggatgattatgcattgtctccacattgtatgcttccaagattctggtgggaatactgctgatagcctaacgttcatgatcaaaatttaactgttctaacccctacttgacagcaatatataaacagaaggaagctgccctgtcttaaacctttttttttatcatcattattagcttactttcataattgcgactggttccaattgacaagcttttgattttaacgacttttaacgacaacttgagaagatcaaaaaacaactaattattcgaaacgatgcatcatcatcatcatcatagctccggtagagagaacttgtacttccaaggtatggccgttaagcatctgatcgttttaaagttcaaggatgagatcaccgaggctcaaaaggaagagtttttcaagacttacgtcaacttggtcaacatcattccagctatgaaggatgtctactggggtaaggacgtcactcaaaagaacaaggaggagggatacacccacatagtggaggttactttcgagtctgtcgagactatccaggactacatcattcacccagcccacgttggattcggcgatgtctacagatctttttgggagaagttgctgatctttgactacactccaagaaaaggttcataatcaagaggatgtcagaatgccatttgcctgagagatgcaggcttcatttttgatacttttttatttgtaacctatatagtataggattttttttgtcattttgtttcttctcgtacgagcttgctcctgatcagcctatctcgcagctgatgaatatcttgtggtaggggtttgggaaaatcattcgagtttgatgtttttcttggtatttcccactcctcttcagagtacagaagattaagtgacacgttcgtttgtgcaagcttcaacgatgccaaaagggtataataagcgtcatttgcagcattgtgaagaaaactatgtggcaagccaagcctgcgaagaatgta

SEQ ID NO: 4 - protein, 341 aa, Cannabis sativa
MHHHHHHSSGRENLYFQGMSAGSDQIEGSPHHESDNSIATKILNFGHTCWKLQRPYVVKGMISIACGLFGRELFNNRHLFSWGLMWKAFFALVPILSFNFFAAIMNQIYDVDIDRINKPDLPLVSGEMSIETAWILSIIVALTGLIVTIKLKSAPLFVFIYIFGIFAGFAYSVPPIRWKQYPFTNFLITISSHVGLAFTSYSATTSALGLPFVWRPAFSFIIAFMTVMGMTIAFAKDISDIEGDAKYGVSTVATKLGARNMTFVVSGVLLLNYLVSISIGIIWPQVFKSNIMILSHAILAFCLIFQTRELALANYASAPSRQFFEFIWLLYYAEYFVYVFI

SEQ ID NO: 5 - protein, 121 aa, Cannabis sativa
MHHHHHHSSGRENLYFQGMAVKHLIVLKFKDEITEAQKEEFFKTYVNLVNIIPAMKDVYWGKDVTQKNKEEGYTHIVEVTFESVETIQDYIIHPAHVGFGDVYRSFWEKLLIFDYTPRKGS

SEQ ID NO: 6 - DNA, 1218 bp, Cannabis sativa
atgcatcatcatcatcatcattcctccggtagagagaacttgtacttccaaggtatgaaccatttgagagctgaaggtcctgcttccgttttggctattggaactgccaatccagagaacatccttttgcaagatgagtttccagactactatttccgtgtcaccaagtctgaacacatgactcaattgaaggagaagttcagaaagatttgtgataagtcaatgatcagaaagagaaactgttttttgaacgaggaacatttgaaacaaaacccaagattagttgagcatgaaatgcaaacattggacgctagacaggatatgttggttgttgaggttccaaagttgggtaaggatgcttgtgccaaggccatcaaggaatggggtcagccaaagtccaagattactcacttgatctttacaagcgcttctaccactgacatgccaggagccgactaccactgcgccaagttgttgggactgtctccttctgttaagagagttatgatgtaccaattgggatgttacggtggtggtaccgttttgagaattgccaaggacattgctgagaacaacaagggtgccagagttttggctgtttgttgtgatatcatggcttgtttgttcagaggaccaagcgaatcagatttggaattgttggttggccaagccatttttggagatggtgctgctgctgttattgttggtgccgaaccagacgaatctgttggagagagaccaatcttcgaattagtttcgactggtcagacaattttgcccaactctgaaggaacaattggtggtcacatcagagaagctggtttgatcttcgacttgcacaaggatgtaccaatgctgatttccaacaacattgagaagtgtttgattgaggcttttacccctattggtatttctgattggaactccatcttctggatcactcatccaggtggtaaggccattttggacaaagttgaggagaagttgcacttgaagtctgataagtttgttgactccagacacgtgctctctgagcacggtaatatgtcatcttccactgttctgttcgtcatggatgagttgagaaagagatctttggaggaaggtaagtctactacaggcgatggatttgaatggggtgttttgttcggtttcgggccaggtttgaccgtcgagagagttgttgttagatctgttccaattaagtacggatcttaa

SEQ ID NO: 7 - protein, 405 aa, Cannabis sativa
MHHHHHHSSGRENLYFQGMNHLRAEGPASVLAIGTANPENILLQDEFPDYYFRVTKSEHMTQLKEKFRKICDKSMIRKRNCFLNEEHLKQNPRLVEHEMQTLDARQDMLVVEVPKLGKDACAKAIKEWGQPKSKITHLIFTSASTTDMPGADYHCAKLLGLSPSVKRVMMYQLGCYGGGTVLRIAKDIAENNKGARVLAVCCDIMACLFRGPSESDLELLVGQAIFGDGAAAVIVGAEPDESVGERPIFELVSTGQTILPNSEGTIGGHIREAGLIFDLHKDVPMLISNNIEKCLIEAFTPIGISDWNSIFWITHPGGKAILDKVEEKLHLKSDKFVDSRHVLSEHGNMSSSTVLFVMDELRKRSLEEGKSTTGDGFEWGVLFGFGPGLTVERVVVRSVPIKYGS

SEQ ID NO: 8 - DNA, 1026 bp, Cannabis sativa
atgcatcatcatcatcaccattcctccggtagagagaacttgtacttccaaggtatgtctgctggttctgaccaaatcgaaggttccccacaccacgagtctgataactccattgctaccaagattttgaactttggccatacgtgttggaaattgcaaagaccttacgttgtcaagggtatgatatcgattgcttgtggtttgtttggtagagagttgttcaacaacagacatttgttctcttggggtttgatgtggaaggctttttttgcccttgttcctattctttctttcaacttttttgccgctatcatgaaccagatttacgatgttgatattgacagaatcaacaagccagaccttccattggtttctggtgagatgtccattgagactgcttggattctgtcaattatagttgctttgactggtttgattgtcactatcaagctgaaatctgctccactgtttgttttcatttacatctttggtatttttgctggttttgcctattcggttcctccaattagatggaagcaatatccattcaccaactttctgatcacaatttcctctcacgttggtttggctttcacaagctactccgctactacctctgctttgggtcttccattcgtgtggcgtccagccttttctttcatcattgctttcatgactgtcatgggtatgaccattgcttttgccaaggacatttctgatatcgaaggtgacgctaagtacggtgtctcgaccgttgctaccaagttgggtgccagaaatatgactttcgttgtttctggtgttttgctgttgaactacctggtgagtatttcaattggtattatctggccccaagtcttcaagtccaacattatgattctatctcacgctattttggctttctgtttgatcttccaaaccagagagctggctttggccaactacgcttccgctccatccagacaattctttgaattcatttggctattgtactacgccgagtacttcgtttatgttttcatctaa

SEQ ID NO: 9 - DNA, 366 bp, Cannabis sativa
atgcatcatcatcatcatcatagctccggtagagagaacttgtacttccaaggtatggccgttaagcatctgatcgttttaaagttcaaggatgagatcaccgaggctcaaaaggaagagtttttcaagacttacgtcaacttggtcaacatcattccagctatgaaggatgtctactggggtaaggacgtcactcaaaagaacaaggaggagggatacacccacatagtggaggttactttcgagtctgtcgagactatccaggactacatcattcacccagcccacgttggattcggcgatgtctacagatctttttgggagaagttgctgatctttgactacactccaagaaaaggttcataa

SEQ ID NO: 10 - DNA, 4005 bp, Cannabis sativa
gatctaacatccaaagacgaaaggttgaatgaaacctttttgccatccgacatccacaggtccattctcacacataagtgccaaacgcaacaggaggggatacactagcagcagaccgttgcaaacgcaggacctccactcctcttctcctcaacacccacttttgccatcgaaaaaccagcccagttattgggcttgattggagctcgctcattccaattccttctattaggctactaacaccatgactttattagcctgtctatcctggcccccctggcgaggttcatgtttgtttatttccgaatgcaacaagctccgcattacacccgaacatcactccagatgagggctttctgagtgtggggtcaaatagtttcatgttccccaaatggcccaaaactgacagtttaaacgctgtcttggaacctaatatgacaaaagcgtgatctcatccaagatgaactaagtttggttcgttgaaatgctaacggccagttggtcaaaaagaaacttccaaaagtcggcataccgtttgtcttgtttggtattgattgacgaatgctcaaaaataatctcattaatgcttagcgcagtctctctatcgcttctgaaccccggtgcacctgtgccgaaacgcaaatggggaaacacccgctttttggatgattatgcattgtctccacattgtatgcttccaagattctggtgggaatactgctgatagcctaacgttcatgatcaaaatttaactgttctaacccctacttgacagcaatatataaacagaaggaagctgccctgtcttaaacctttttttttatcatcattattagcttactttcataattgcgactggttccaattgacaagcttttgattttaacgacttttaacgacaacttgagaagatcaaaaaacaactaattattcgaaacgatgcatcatcatcatcaccattcctccggtagagagaacttgtacttccaaggtatgtctgctggttctgaccaaatcgaaggttccccacaccacgagtctgataactccattgctaccaagattttgaactttggccatacgtgttggaaattgcaaagaccttacgttgtcaagggtatgatatcgattgcttgtggtttgtttggtagagagttgttcaacaacagacatttgttctcttggggtttgatgtggaaggctttttttgcccttgttcctattctttctttcaacttttttgccgctatcatgaaccagatttacgatgttgatattgacagaatcaacaagccagaccttccattggtttctggtgagatgtccattgagactgcttggattctgtcaattatagttgctttgactggtttgattgtcactatcaagctgaaatctgctccactgtttgttttcatttacatctttggtatttttgctggttttgcctattcggttcctccaattagatggaagcaatatccattcaccaactttctgatcacaatttcctctcacgttggtttggctttcacaagctactccgctactacctctgctttgggtcttccattcgtgtggcgtccagccttttctttcatcattgctttcatgactgtcatgggtatgaccattgcttttgccaaggacatttctgatatcgaaggtgacgctaagtacggtgtctcgaccgttgctaccaagttgggtgccagaaatatgactttcgttgtttctggtgttttgctgttgaactacctggtgagtatttcaattggtattatctggccccaagtcttcaagtccaacattatgattctatctcacgctattttggctttctgtttgatcttccaaaccagagagctggctttggccaactacgcttccgctccatccagacaattctttgaattcatttggctattgtactacgccgagtacttcgtttatgttttcatctaatgaaatgtatttaatttgatattaagtaaatgaatgattatgactttatgaattcgcaatgttttctccttgattatttctgtattgtattggaatgattatagaatactcatatattgattatagtattagcacataaaacgtttgttgttaaactcacttccgtacgcaaccatttctatttctagctatcttgataaggtgatctaacatccaaagacgaaaggttgaatgaaacctttttgccatccgacatccacaggtccattctcacacataagtgccaaacgcaacaggaggggatacactagcagcagaccgttgcaaacgcaggacctccactcctcttctcctcaacacccacttttgccatcgaaaaaccagcccagttattgggcttgattggagctcgctcattccaattccttctattaggctactaacaccatgactttattagcctgtctatcctggcccccctggcgaggttcatgtttgtttatttccgaatgcaacaagctccgcattacacccgaacatcactccagatgagggctttctgagtgtggggtcaaatagtttcatgttccccaaatggcccaaaactgacagtttagagacctattccagacttcagatctccaaaaacaggactatatcataatttggcacgtttgaatcttccccatcctgaagcggtatttgagatcaactacttcagagaaaaccctcatgctttttacacattggctgacgaactttaccctgggagggtctcaaaacgctgtcttggaacctaatatgacaaaagcgtgatctcatccaagatgaactaagtttggttcgttgaaatgctaacggccagttggtcaaaaagaaacttccaaaagtcggcataccgtttgtcttgtttggtattgattgacgaatgctcaaaaataatctcattaatgcttagcgcagtctctctatcgcttctgaaccccggtgcacctgtgccgaaacgcaaatggggaaacacccgctttttggatgattatgcattgtctccacattgtatgcttccaagattctggtgggaatactgctgatagcctaacgttcatgatcaaaatttaactgttctaacccctacttgacagcaatatataaacagaaggaagctgccctgtcttaaacctttttttttatcatcattattagcttactttcataattgcgactggttccaattgacaagcttttgattttaacgacttttaacgacaacttgagaagatcaaaaaacaactaattattcgaaacgatgcatcatcatcatcatcatagctccggtagagagaacttgtacttccaaggtatggccgttaagcatctgatcgttttaaagttcaaggatgagatcaccgaggctcaaaaggaagagtttttcaagacttacgtcaacttggtcaacatcattccagctatgaaggatgtctactggggtaaggacgtcactcaaaagaacaaggaggagggatacacccacatagtggaggttactttcgagtctgtcgagactatccaggactacatcattcacccagcccacgttggattcggcgatgtctacagatctttttgggagaagttgctgatctttgactacactccaagaaaaggttcataatcaagaggatgtcagaatgccatttgcctgagagatgcaggcttcatttttgatacttttttatttgtaacctatatagtataggattttttttgtcattttgtttcttctcgtacgagcttgctcctgatcagcctatctcgcagctgatgaatatcttgtggtaggggtttgggaaaatcattcgagtttgatgtttttcttggtatttcccactcctcttcagagtacagaagattaagtgacacgttcgtttgtgcaagcttcaacgatgccaaaagggtataataagcgtcatttgcagcattgtgaagaaaactatgtggcaagccaagcctgcgaagaatgtagtcgagaattgagcttgc